(19) United States
(12) Patent Application Publication
(10) Pub. No.: US 2009/0324647 A1
(43) Pub. Date: Dec. 31, 2009

(54) Albumin-Free Botulinum Toxin Based Pharmaceutical Compositions Containing a Hyaluronidase and Methods of Use

(76) Inventor: Gary E. Borodic, Quincy, MA (US)

Correspondence Address: MILBANK, TWEED, HADLEY & MCCLOY LLP, INTERNATIONAL SQUARE BUILDING, 1850 K STRET, N.W., SUITE 1100, WASHINGTON, DC, 20006, US

(21) Appl. No.: 12/083,058

(22) PCT Filed: Oct. 11, 2006
(86) PCT No.: PCT/US2006/039723

§ 371 Date: Aug. 3, 2009

Related U.S. Application Data
(60) Provisional application No. 60/724,913, filed on Oct. 11, 2005.

(51) Int. Cl. A61K 39/08; A61P 21/00; A61P 29/00; A61P 25/00

(52) U.S. Cl. 424/239.1

(57) ABSTRACT

The present invention provides compositions that contain botulinum toxin and a hyaluronidase, and that lack human or recombinant serum albumin. The present invention also provides methods of administering the pharmaceutical composition to a subject in need thereof.

### TECHNICAL FIELD OF THE INVENTION

This invention relates to pharmaceutical formulations of botulinum toxin lacking human serum albumin. This invention further relates to pharmaceutical compositions of botulinum toxin and hyaluronidase. The invention further relates to methods for the treatment of a variety of neuromuscular diseases, pain, inflammatory and cutaneous disorders with botulinum toxin formulations. The present invention also relates to the treatment of primary disorders of mood and affect using the pharmaceutical compositions disclosed herein, including depressive, anxiety and sleep disorders as well as other CNS disorders.

### BACKGROUND OF THE INVENTION

### A. Botulinum Toxin: Mechanism of Action

Botulinum neurotoxin is a toxin isolated from a strain of Clostridium botulinum, that acts at the neuromuscular junction by inhibiting release of acetylcholine. Botulinum toxin is initially formed as a single-chain polypeptide that is cleaved to form a light chain that is bound to a heavy chain through a disulfide bond. The denervating effect of botulinum toxin occurs through: 1) the binding of the heavy chain to high-affinity receptors at the presynaptic terminal; 2) internalization of botulinum toxin through endocytosis; 3) translocation of the light chain into the cytoplasm of the nerve terminal; and 4) the endo-metalloprotease activity of the light chain (zinc cofactor) cleaves specific synaptic proteins that inhibit fusion of synaptic vesicles with the presynaptic membrane, thereby inhibiting the release of acetylcholine contained in the vesicles. Absent acetylcholine, the muscle does not receive the necessary signal for the muscle to contract. Subsequent to injection, neurogenic muscular atrophy ensues after several weeks.

### B. Botulinum Toxin: Clinical Applications

A deadly toxin at high concentrations and quantities, botulinum toxin has been used as a valuable therapeutic for the treatment of many neuromuscular diseases (e.g., dystonia, hemifacial spasm, bruxism, spasticity, cerebral palsy, torticollis), as well as sensory disorders and cutaneous disorders (myofascial pain, migraine, tension headaches, neuropathy, hyperhydrosis), and in the treatment of disorders involving inflammation. The therapeutic value of botulinum toxin is in its ability to produce local regional denervation of specific muscles and tissues.

The action of botulinum toxin on nerve terminals is irreversible. Axon sprouting, however, reverses the denervating effects of the toxin within two to six months. Consequently, a variety of conditions and disorders require repeated administration of the neurotoxin. Resistance to botulinum toxin is an important clinical consequence and problem resulting from repeated administration of botulinum toxin and the production of neutralizing antibodies. (Naumann et al. (1998) J. Neurol. Neurosurg. Psychiatry 65: 924-927; Hauna et al. (1998) J. Neurol. Neurosurg. Psychiatry 66: 612-616).

Botulinum based pharmaceuticals currently available have been formulated with human serum albumin in order to provide stability during dilution, lyophilization (drying) and storage. As human albumin is noted to be one of the least likely excipient proteins to elicit an immunologic reaction, formulating botulinum toxin with human serum albumin has generally proved effective and safe during past years. Recently, with the growing concern regarding prion-based progressive spongiform encephalopathy (mad cow disease), methods and approaches have been taken to eliminate blood components in commercially-available pharmaceuticals. Recently, one blood born case of Creuztfeld-Jacob disease has been reported in England and thought to be blood born (blood donor and recipient both developing the disease within a short time period).

Attempts at manufacturing recombinant human serum albumin have been fraught with problems of immunogenicity. The compositions of the present invention comprise unique formulations of botulinum toxin which excludes the use of human or recombinant serum albumin and uses a source of stabilizing protein not originating from human or cow.

### SUMMARY OF THE INVENTION

This invention relates to compositions of botulinum toxin lacking human serum albumin. This invention further relates to compositions of botulinum toxin and hyaluronidase. The invention further relates to methods for the treatment of a variety of neuromuscular diseases, pain, inflammatory and cutaneous disorders with botulinum toxin formulations. The present invention also relates to the treatment of primary disorders of mood and affect using the pharmaceutical compositions disclosed herein, including depressive, anxiety and sleep disorders as well as other CNS disorders.

The compositions of the present invention comprise a botulinum toxin wherein the botulinum toxin may be selected from any one or a combination of the various botulinum toxin immunotypes such as A, B, C₁, C₂, C₃, D, E, F and G. In a preferred embodiment, the botulinum neurotoxin is botulinum toxin type A. The compositions of the present invention may further comprise a stabilization or stabilizing agent that stabilizes the activity of the botulinum neurotoxin. As used herein, “stabilization agent” or “stabilizing agent” means any agent that prolongs the biologic activity, or specifically the neurotoxicity of the botulinum neurotoxin, upon storage. In a preferred embodiment, the stabilization or stabilizing agent is a monosaccharide or disaccharide. In a more preferred embodiment, lactose, sucrose or trehalose is the stabilization or stabilizing agent. Most preferably, trehalose is the stabilization or stabilizing agent.

The compositions of the present invention comprise a botulinum toxin wherein the botulinum toxin may be of any purity, as described by specific activity or specific neurotoxicity. In a preferred embodiment, the botulinum toxin has a specific neurotoxicity of between about 20 and 250 Units/ng neurotoxin, about 50 and 250 Units/ng neurotoxin, about 80 and 250 Units/ng neurotoxin, about 90 and 250 Units/ng neurotoxin, about 100 and 250 Units/ng neurotoxin, about 150 and 250 Units/ng neurotoxin, or about 200 and 250 Units/ng neurotoxin. In a more preferred embodiment, the botulinum toxin has a specific neurotoxicity of about 20 Units/ng neurotoxin, 30 Units/ng neurotoxin, 40 Units/ng neurotoxin, 50 Units/ng neurotoxin, 60 Units/ng neurotoxin, 70 Units/ng neurotoxin, 80 Units/ng neurotoxin, 90 Units/ng neurotoxin, 100 Units/ng neurotoxin, 110 Units/ng neurotoxin, 120 Units/ng neurotoxin, 130 Units/ng neurotoxin, 140 Units/ng neurotoxin, 150 Units/ng neurotoxin, 160 Units/ng neurotoxin, 170 Units/ng neurotoxin, 180 Units/ng neurotoxin, 190 Units/ng neurotoxin, 200 Units/ng neurotoxin, 210 Units/ng neurotoxin, 220 Units/ng neurotoxin, 230 Units/ng neurotoxin, 240 Units/ng neurotoxin, or 250 Units/ng neurotoxin.

In certain embodiments, the compositions of the invention may be formulated such that when administered, from about 5 LD₅₀ units to about 10,000 LD₅₀ units may be administered per injection to a subject.

Preferably, the compositions may be administered such that from about 10 LD₅₀ units to about 10,000 LD₅₀ units; or about 20 LD₅₀ units to about 10,000 LD₅₀ units; or about 30 LD₅₀ units to about 10,000 LD₅₀ units; or about 40 LD₅₀ units to about 10,000 LD₅₀ units; or about 50 LD₅₀ units to about 10,000 LD₅₀ units; or about 75 LD₅₀ units to about 10,000 LD₅₀ units; or about 100 LD₅₀ units to about 10,000 LD₅₀ units; or about 150 LD₅₀ units to about 10,000 LD₅₀ units; or about 200 LD₅₀ units to about 10,000 LD₅₀ units; or about 250 LD₅₀ units to about 10,000 LD₅₀ units; or about 300 LD₅₀ units to about 10,000 LD₅₀ units; or about 350 LD₅₀ units to about 10,000 LD₅₀ units; or about 400 LD₅₀ units to about 10,000 LD₅₀ units; or about 450 LD₅₀ units to about 10,000 LD₅₀ units; or about 500 LD₅₀ units to about 10,000 LD₅₀ units; or about 550 LD₅₀ units to about 10,000 LD₅₀ units; or about 600 LD₅₀ units to about 10,000 LD₅₀ units; or about 650 LD₅₀ units to about 10,000 LD₅₀ units; or about 700 LD₅₀ units to about 10,000 LD₅₀ units; or about 750 LD₅₀ units to about 10,000 LD₅₀ units; or about 800 LD₅₀ units to about 10,000 LD₅₀ units; or about 850 LD₅₀ units to about 10,000 LD₅₀ units; or about 900 LD₅₀ units to about 10,000 LD₅₀ units; or about 950 LD₅₀ units to about 10,000 LD₅₀ units; or about 1000 LD₅₀ units to about 10,000 LD₅₀ units; or about 1200 to about 10,000 LD₅₀ units; or about 1400 LD₅₀ units to about 10,000 LD₅₀ units; or about 1600 LD₅₀ units to about 10,000 LD₅₀ units; or about 1800 LD₅₀ units to about 10,000 LD₅₀ units; or about 2000 LD₅₀ units to about 10,000 LD₅₀ units or about 2200 LD₅₀ units to about 10,000 LD₅₀ units; or about 2400 LD₅₀ units to about 10,000 LD₅₀ units; or about 2500 LD₅₀ units to about 10,000 LD₅₀ units may be administered per injection to a subject.

In another embodiment of the present invention, the compositions are essentially free of salt. More preferably, the compositions contains less than about 0.9% salt.

Currently, hyaluronidase is available at a number of specific activities. For example, sheep based materials can have a specific activity of 1,500 U per mg or 1.5 U per microgram. Typically, 75-300 U are used for injection, such as conducted with peri-bulbar anesthesia for intra-ocular surgery. This would correspond to about 100-450 mg in mass of enzymatic protein, enough to act as a stabilizing excipient. Recombinantly produced hyaluronidase has a much higher specific activity than non-recombinant hyaluronidase. The compositions of the present invention may comprise from about 100 units hyaluronidase per injection, per vial or per mL to about 500 million units hyaluronidase per injection, per vial or per mL.

Within the scope of the present invention, any amount of hyaluronidase may be combined with any amount of botulinum toxin as disclosed herein and such combinations may be expressed in various ways including, but not limited to units hyaluronidase per LD₅₀ units botulinum toxin; mass hyaluronidase per LD₅₀ units botulinum toxin; mass hyaluronidase per vial; mass hyaluronidase per injection; mass hyaluronidase/volume solution per LD₅₀ units botulinum toxin (eg. mg hyaluronidase/mL per LD₅₀ units botulinum toxin); or as ranges of values or as amounts greater than or less than specified values, etc.

In preferred embodiments, the compositions may comprise about 25; about 50; about 75; about 100; about 500; about 1000; about 1500; about 2000; about 2500; about 3000; about 3500; about 4000; about 4500; about 5000; about 5500; about 6000; about 6500; about 7000; about 7500; about 8000; about 8500; about 9000; about 9500; about 10,000; about 20,000; about 30,000; about 40,000; about 50,000; about 60,000; about 70,000; about 80,000; about 90,000; about 100,000; about 120,000; about 140,000; about 160,000; about 180,000; about 200,000; about 300,000; about 400,000; about 500,000; about 600,000; about 700,000; about 800,000; about 900,000 about 1,000,000; about 2,000,000; about 3,000,000; about 4,000,000; about 5,000,000, about 6,000,000; about 7,000,000; about 8,000,000; about 9,000,000; about 10,000,000; about 20,000,000; about 30,000,000; about 40,000,000; about 50,000,000; about 60,000,000; about 70,000,000; about 80,000,000; about 90,000,000; about 100,000,000; about 200,000,000; about 300,000,000; about 400,000,000 or about 500,000,000 units hyaluronidase per injection, or units hyaluronidase per vial, or units hyaluronidase per mL or units hyaluronidase per LD₅₀ units botulinum toxin.

In other preferred embodiments, the compositions comprise about 100 to about 500; or about 100 to about 1000; or about 100 to about 10,000; or about 100 to about 100,000; or about 100 to about 1,000,000; or about 100 to about 10,000,000; or about 100 to about 100,000,000 or about 100 to about 500,000,000 units hyaluronidase per injection, or units hyaluronidase per vial, or units hyaluronidase per mL or units hyaluronidase per LD₅₀ units botulinum toxin.

In other preferred embodiments, the compositions of the invention comprise about 500 to about 1000; or about 500 to about 10,000; or about 500 to about 100,000; or about 500 to about 1,000,000; or about 500 to about 10,000,000; or about 500 to about 100,000,000 or about 500 to about 500,000,000 units hyaluronidase per injection, or units hyaluronidase per vial, or units hyaluronidase per mL or units hyaluronidase per LD₅₀ units botulinum toxin.

In other preferred embodiments, the compositions of the invention comprise about 1000 to about 10,000; or about 1000 to about 100,000; or about 1000 to about 1,000,000; or about 1000 to about 10,000,000; or about 1000 to about 100,000,000 or about 1000 to about 500,000,000 units hyaluronidase per injection, or units hyaluronidase per vial, or units hyaluronidase per mL or units hyaluronidase per LD₅₀ units botulinum toxin.

In certain embodiments, the compositions of the invention may comprise from greater than about 1 nanogram (ng) to greater than about 1 gram hyaluronidase per injection or per vial or per mL or per LD₅₀ units botulinum toxin or (unit mass hyaluronidase/mL per LD₅₀ units botulinum toxin).

In preferred embodiments, the compositions of the invention may comprise greater than about 1 ng, greater than about 5 ng; greater than about 10 ng; greater than about 15 ng; greater than about 20 ng; greater than about 25 ng; greater than about 30 ng; greater than about 35 ng; greater than about 40 ng; greater than about 45 ng; greater than about 50 ng; greater than about 60 ng; greater than about 70 ng; greater than about 80 ng; greater than about 90 ng; greater than about 100 ng; greater than about 120 ng; greater than about 140 ng; greater than about 160 ng; greater than about 180 ng; greater than about 200 ng; greater than about 220 ng; greater than about 240 ng; greater than about 260 ng; greater than about 280 ng; greater than about 300 ng; greater than about 320 ng; greater than about 340 ng; greater than about 360 ng; greater than about 380 ng; greater than about 400 ng; greater than about 420 ng; greater than about 440 ng; greater than about 460 ng; greater than about 480 ng; greater than about 500 ng; greater than about 520 ng; greater than about 540 ng; greater than about 560 ng; greater than about 580 ng; greater than about 600 ng; greater than about 620 ng; greater than about 640 ng; 660 ng; greater than about 680 ng; greater than about 700 ng; greater than about 720 ng; greater than about 740 ng; greater than about 760 ng; greater than about 780 ng; greater than about 800 ng; greater than about 820 ng; greater than about 840 ng; greater than about 860 ng; greater than about 880 ng; greater than about 900 ng; greater than about 920 ng; greater than about 940 ng; greater than about 960 ng; greater than about 980 ng; greater than about 1 microgram, greater than about 5 microgram; greater than about 10 microgram; greater than about 15 microgram; greater than about 20 microgram; greater than about 25 microgram; greater than about 30 microgram; greater than about 35 microgram; greater than about 40 microgram; greater than about 45 microgram; greater than about 50 microgram; greater than about 60 microgram; greater than about 70 microgram; greater than about 80 microgram; greater than about 90 microgram; greater than about 100 microgram; greater than about 120 microgram; greater than about 140 microgram; greater than about 160 microgram; greater than about 180 microgram; greater than about 200 microgram; greater than about 220 microgram; greater than about 240 microgram; greater than about 260 microgram; greater than about 280 microgram; greater than about 300 microgram; greater than about 320 microgram; greater than about 340 microgram; greater than about 360 microgram; greater than about 380 microgram; greater than about 400 microgram; greater than about 420 microgram; greater than about 440 microgram; greater than about 460 microgram; greater than about 480 microgram; greater than about 500 microgram; greater than about 520 microgram; greater than about 540 microgram; greater than about 560 microgram; greater than about 580 microgram; greater than about 600 microgram; greater than about 620 microgram; greater than about 640 microgram; 660 microgram; greater than about 680 microgram; greater than about 700 microgram; greater than about 720 microgram; greater than about 740 microgram; greater than about 760 microgram; greater than about 780 microgram; greater than about 800 microgram; greater than about 820 microgram; greater than about 840 microgram; greater than about 860 microgram; greater than about 880 microgram; greater than about 900 microgram; greater than about 920 microgram; greater than about 940 microgram; greater than about 960 microgram; greater than about 980 microgram; greater than about 1 mg, greater than about 5 mg; greater than about 10 mg; greater than about 15 mg; greater than about 20 mg; greater than about 25 mg; greater than about 30 mg; greater than about 35 mg; greater than about 40 mg; greater than about 45 mg; greater than about 50 mg; greater than about 60 mg; greater than about 70 mg; greater than about 80 mg; greater than about 90 mg; greater than about 100 mg; greater than about 120 mg; greater than about 140 mg; greater than about 160 mg; greater than about 180 mg; greater than about 200 mg; greater than about 220 mg; greater than about 240 mg; greater than about 260 mg; greater than about 280 mg; greater than about 300 mg; greater than about 320 mg; greater than about 340 mg; greater than about 360 mg; greater than about 380 mg; greater than about 400 mg; greater than about 420 mg; greater than about 440 mg; greater than about 460 mg; greater than about 480 mg; greater than about 500 mg; greater than about 520 mg; greater than about 540 mg; greater than about 560 mg; greater than about 580 mg; greater than about 600 mg; greater than about 620 mg; greater than about 640 mg; 660 mg; greater than about 680 mg; greater than about 700 mg; greater than about 720 mg; greater than about 740 mg; greater than about 760 mg; greater than about 780 mg; greater than about 800 mg; greater than about 820 mg; greater than about 840 mg; greater than about 860 mg; greater than about 880 mg; greater than about 900 mg; greater than about 920 mg; greater than about 940 mg; greater than about 960 mg; greater than about 980 mg or greater than about 1 gram hyaluronidase per injection or per vial or per mL or per LD₅₀ units botulinum toxin or (unit mass hyaluronidase/mL per LD₅₀ units botulinum toxin).

In still further preferred embodiments, the compositions of the invention may comprise from about 1 ng to about 1 microgram, or about 1 ng to about 5 microgram; or about 1 ng to about 10 microgram; or about 1 ng to about 15 microgram; or about 1 ng to about 20 microgram; or about 1 ng to about 25 microgram; or about 1 ng to about 30 microgram; or about 1 ng to about 35 microgram; or about 1 ng to about 40 microgram; or about 1 ng to about 45 microgram; or about 1 ng to about 50 microgram; or about 1 ng to about 60 microgram; or about 1 ng to about 70 microgram; or about 1 ng to about 80 microgram; or about 1 ng to about 90 microgram; or about 1 ng to about 100 microgram; or about 1 ng to about 120 microgram; or about 1 ng to about 140 microgram; or about 1 ng to about 160 microgram; or about 1 ng to about 180 microgram; or about 1 ng to about 200 microgram; or about 1 ng to about 220 microgram; or about 1 ng to about 240 microgram; or about 1 ng to about 260 microgram; or about 1 ng to about 280 microgram; or about 1 ng to about 300 microgram; or about 1 ng to about 320 microgram; or about 1 ng to about 340 microgram; or about 1 ng to about 360 microgram; or about 1 ng to about 380 microgram; or about 1 ng to about 400 microgram; or about 1 ng to about 420 microgram; or about 1 ng to about 440 microgram; or about 1 ng to about 460 microgram; or about 1 ng to about 480 microgram; or about 1 ng to about 500 microgram; or about 1 ng to about 520 microgram; or about 1 ng to about 540 microgram; or about 1 ng to about 560 microgram; or about 1 ng to about 580 microgram; or about 1 ng to about 600 microgram; or about 1 ng to about 620 microgram; or about 1 ng to about 640 microgram; or about 1 ng to 660 microgram; or about 1 ng to about 680 microgram; or about 1 ng to about 700 microgram; or about 1 ng to about 720 microgram; or about 1 ng to about 740 microgram; or about 1 ng to about 760 microgram; or about 1 ng to about 780 microgram; or about 1 ng to about 800 microgram; or about 1 ng to about 820 microgram; or about 1 ng to about 840 microgram; or about 1 ng to about 860 microgram; or about 1 ng to about 880 microgram; or about 1 ng to about 900 microgram; or about 1 ng to about 920 microgram; or about 1 ng to about 940 microgram; or about 1 ng to about 960 microgram; or about 1 ng to about 980 microgram; or about 1 ng to about 1 mg, or about 1 ng to about 5 mg; or about 1 ng to about 10 mg; or about 1 ng to about 15 mg; or about 1 ng to about 20 mg; or about 1 ng to about 25 mg; or about 1 ng to about 30 mg; or about 1 ng to about 35 mg; or about 1 ng to about 40 mg; or about 1 ng to about 45 mg; or about 1 ng to about 50 mg; or about 1 ng to about 60 mg; or about 1 ng to about 70 mg; or about 1 ng to about 80 mg; or about 1 ng to about 90 mg; or about 1 ng to about 100 mg; or about 1 ng to about 120 mg; or about 1 ng to about 140 mg; or about 1 ng to about 160 mg; or about 1 ng to about 180 mg; or about 1 ng to about 200 mg; or about 1 ng to about 220 mg; or about 1 ng to about 240 mg; or about 1 ng to about 260 mg; or about 1 ng to about 280 mg; or about 1 ng to about 300 mg; or about 1 ng to about 320 mg; or about 1 ng to about 340 mg; or about 1 ng to about 360 mg; or about 1 ng to about 380 mg; or about 1 ng to about 400 mg; or about 1 ng to about 420 mg; or about 1 ng to about 440 mg; or about 1 ng to about 460 mg; or about 1 ng to about 480 mg; or about 1 ng to about 500 mg; or about 1 ng to about 520 mg; or about 1 ng to about 540 mg; or about 1 ng to about 560 mg; or about 1 ng to about 580 mg; or about 1 ng to about 600 mg; or about 1 ng to about 620 mg; or about 1 ng to about 640 mg; or about 1 ng to 660 mg; or about 1 ng to about 680 mg; or about 1 ng to about 700 mg; or about 1 ng to about 720 mg; or about 1 ng to about 740 mg; or about 1 ng to about 760 mg; or about 1 ng to about 780 mg; or about 1 ng to about 800 mg; or about 1 ng to about 820 mg; or about 1 ng to about 840 mg; or about 1 ng to about 860 mg; or about 1 ng to about 880 mg; or about 1 ng to about 900 mg; or about 1 ng to about 920 mg; or about 1 ng to about 940 mg; or about 1 ng to about 960 mg; or about 1 ng to about 980 mg; or about 1 ng to or about 1 gram hyaluronidase per injection or per vial or per mL or per LD50 units botulinum toxin, or (unit mass range/mL per LD₅₀ units botulinum toxin).

In one embodiment of the present invention, the compositions have a pH of between about 5.8 to 7.4, about 6 to 7.4, about 6.2 to 7.4, about 6.5 to 7.4, about 6.7 to 7.4, about 7 to 7.4, or about 7.2 to 7.4. Preferably, the compositions have a pH of about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, or about 7.4.

The compositions of the present invention may be administered by any means known in the art sufficient to deliver the botulinum toxin to the desired therapeutic target. Preferably, the compositions are delivered by transmucosal administration, transcutaneous administration, intramuscular administration or topically. Preferably, the compositions of the present invention are administered by injection, including by needle, micro-needle or needleless injection.

The compositions of the present invention may be used in any of the methods of treatment disclosed herein. According to the inventive methods described herein, the compositions of the present invention may be administered as a single treatment or repeated periodically to provide multiple treatments.

The present invention also provides methods for muscle denervation comprising the step of administering any of the compositions of the present invention to a subject in need thereof in an amount sufficient to produce local muscle denervation. In another embodiment, the compositions are administered to the muscles of a head, face, eye, neck, back, or tissues overlying one or more nasal sinuses.

In another embodiment, the present invention provides methods for treating neuromuscular diseases comprising the step of administering any of the compositions of the present invention to a subject in need thereof in an amount sufficient to produce muscle weakness. In another embodiment, the neuromuscular disease is cervical dystonia, hemifacial spasm, bruxism, blepharospasm, strabismus, or muscle spasticity. In a preferred embodiment, the neuromuscular disease hemifacial spasm, cervical dystonia, blepharospasm, strabismus, or muscle spasticity.

In one embodiment, the neuromuscular disease is hemifacial spasm, cervical dystonia, blepharospasm, strabismus, or muscle spasticity.

In another embodiment, the present invention provides methods for treating pain comprising the step of administering any of the compositions of the present invention to a subject in need thereof in an amount sufficient to reduce pain. In another embodiment, the patient suffers from myofascial pain, migraine headache pain, tension headache pain, neuropathic pain, facial pain, lower-back pain, sinus-headache pain, pain associated with temporomandibular joint disease, pain associated with spasticity or cervical dystonia, post-surgical wound pain, or neuralgia.

In a preferred embodiment, the patient suffers from sinus headache pain or facial pain associated with acute or recurrent chronic sinusitis. Preferably, any of the compositions of the present invention may be administered to the nasal mucosa or to the subcutaneous structures overlying the sinuses, wherein the administration of the formulation reduces the headache and/or facial pain associated with acute recurrent or chronic sinusitis. More preferably, any of the compositions of the present invention may be administered to the nasal mucosa. The subcutaneous structures overlying the sinuses preferably overly one or more of the sinuses selected from the group consisting of: ethmoid; maxillary; mastoid; frontal; and sphenoid. In another embodiment, subcutaneous structures overlying the sinuses lie within one or more of the areas selected from the group consisting of: forehead; malar; temporal; post auricular; and lip.

In another embodiment, a patient suffering from sinus headache pain or facial pain associated with acute or recurrent chronic sinusitis is treated by administering any of the compositions of the present invention to an afflicted area of the patient. In a preferred embodiment, the compositions disclosed herein are administered to the projections of a trigeminal nerve innervating a sinus.

Patients suffering from sinus headache pain or facial pain associated with acute or recurrent chronic sinusitis often exhibit symptoms including rhinitis, sinus hypersecretion and/or purulent nasal discharge. In one embodiment, the patients treated with the compositions of the present invention exhibit symptoms of sinus hypersecretion and purulent nasal discharge.

The present invention also provides methods for treating a patient suffering from sinus headache pain or facial pain associated with acute or recurrent chronic sinusitis, wherein the subject suffers from neuralgia. Preferably, the neuralgia is trigeminal neuralgia. In another embodiment, the neuralgia is: associated with compressive forces on a sensory nerve; associated with intrinsic nerve damage, demyelinating disease, or a genetic disorder; associated with a metabolic disorder; associated with central neurologic vascular disease; or associated with trauma. In another embodiment of the present invention, the pain is associated with dental extraction or reconstruction.

In another embodiment, the present invention provides methods for cosmetically modifying soft-tissue features comprising the step of administering any of the compositions of the present invention to a subject in need thereof in an amount sufficient to modify said features. In a preferred embodiment, the composition is administered via transcutaneous or transmucosal injection either at a single focus or multiple foci.

Preferably, the compositions of the present invention are administered to the face or neck of the subject. In a preferred embodiment, the compositions of the present invention are administered to the subject in an amount sufficient to reduce rhytides. Preferably, the formulation is administered between eyebrows of the subject in an amount sufficient to reduce vertical lines between the eyebrows and on a bridge of a nose. The compositions may also be administered near either one or both eyes of the subject in an amount sufficient to reduce lines at corners of the eyes. In another embodiment, the compositions of the present invention may also be administered to a forehead of the subject in an amount sufficient to reduce horizontal lines on said forehead. In yet another embodiment of the present invention the composition is administered to the neck of the subject in an amount sufficient to reduce muscle bands in the neck.

The present invention provides methods for reducing lip volume in one or both of the upper and lower lips of a patient. In one embodiment, the patient suffers from hypervolemic lip deformity. Preferably, the compositions of the present invention are administered to a orbicularis oris muscle of the subject. The pharmaceutical botulinum toxin formulations of the present invention may also be administered to one or more lip retractor muscle.

In another embodiment, the present invention provides methods for treating inflammation comprising the step of administering any of the compositions of the present invention to a subject in need thereof in an amount sufficient to reduce inflammation. Preferably, the compositions of the present invention are administered to a patient without producing muscle weakness. In one embodiment, the compositions of the present invention are administered to patients with an inflammatory condition. Preferably, the inflammatory condition is neurogenic inflammation. In another embodiment, the subject suffers from rheumatoid arthritis or a gastro-intestinal inflammatory disease.

In another embodiment, the present invention provides methods for treating cutaneous disorders comprising the step of administering any of the compositions of the present invention to a subject in need thereof in an amount sufficient to reduce a sebaceous or mucous secretion. Preferably, the compositions of the present invention are administered to a patient without producing muscle weakness. In one embodiment, the compositions of the present invention are administered to patients with chalazion or hordeola. Preferably, the compositions of the present invention are injected into one or more sites of an eyelid or conjunctiva. In another embodiment, the formulations of the present invention are administered to a body surface. In another embodiment, the compositions are administered in an amount sufficient to reduce cutaneous bacterial or fungal growth, including but not limited to Staphylococcus; Streptococcus and Moraxella. Preferably, the compositions of the present invention are administered to an area selected from the group consisting of: eyelid; scalp; feet; groin; and armpit to reduce cutaneous infection.

In another embodiment, the cutaneous disorder is hyperhydrosis.

The present invention also provides methods for treating inflammation comprising the step of administering any of the compositions of the present invention to a subject in need thereof in an amount sufficient to reduce inflammation. Preferably, the compositions of the present invention are administered to a patient without producing muscle weakness. In one embodiment, the compositions of the present invention are administered to patients with an inflammatory condition. Preferably, the inflammatory condition is neurogenic inflammation. In another embodiment, the subject suffers from rheumatoid arthritis or a gastrointestinal inflammatory disease.

In a preferred embodiment, the composition comprising a botulinum toxin is administered to a subject suffering muscle spasticity in the flexor digitorum profundus muscle or the flexor digitorum sublimus muscle.

The present invention also provides methods for reducing scarring and/or cosmetic deformity associated with burns or skin disorders such as blistering dermatosis comprising the step of administering a composition comprising a botulinum toxin to a subject in need thereof, wherein administration of said formulation reduces scarring and/or cosmetic deformity. Further, the composition of the present invention may comprise one or more agents that promote cutaneous absorption and penetration. In a preferred embodiment, the composition is administered to a body surface of said subject. In a preferred embodiment, the composition is administered as a liquid formulation. More preferably, the composition is applied as an aerosol. In another embodiment, between about 1 and 10, about 1 and 50, about 1 and 100, about 1 and 200, about 1 and 500, about 1 and 1000, about 1 and 1250, about 1 and 1500, about 1 and 2000, or about 1 and 2500 Units per treatment are administered to a subject suffering from a burn. As used herein, “burn” includes but is not limited to thermal, electrical, or chemical burns and also includes blistering caused by dermatitis and other blistering disorders. In an alternative embodiment, mechanical abrasion, chemical, thermal, laser-induced disruption of skin barriers, and the like, may be used to improve the delivery of topical administration of compositions of botulinum toxin.

In another embodiment, the compositions of the present invention comprise a botulinum toxin that consists essentially of fractionated-light-chain botulinum toxin. In yet another embodiment, the botulinum toxin consists essentially of a mixture of hybrid and chain-translocated forms of botulinum toxin. In a further embodiment, the botulinum toxin consists essentially of chimeric forms of botulinum toxin. Although the present invention may utilize any botulinum toxin, botulinum toxin fragment that retains neurotoxic activity, botulinum toxin chimeras and hybrids, chemically-modified botulinum toxin, and specific activities well known to those of ordinary skill in the art, in one embodiment the botulinum toxin is purified to a specific activity greater than or equal to 20 LD₅₀ units per nanogram botulinum toxin.

Each composition of the present invention, may further comprise a pharmaceutically acceptable carrier and/or zinc and/or a zinc salt. In one embodiment, the botulinum toxin is noncovalently bound to the hyaluronidase. In another embodiment, the botulinum toxin is covalently bound to the hyaluronidase.

The present invention also provides methods of producing localized denervation in a subject in need thereof, comprising administering an effective amount of any of the compositions of the present invention that are described herein. In one embodiment, the methods of the present invention are used to produce denervation in a subject that suffers from a neuromuscular disease associated with increased muscle tone with involuntary movement. In another embodiment, the methods of the present invention are used to produce denervation in a subject that suffers from a neuromuscular disease. Preferably, the neuromuscular disease is characterized by increased muscle tone and/or involuntary movement, including but not limited to dystonias, spinal cord injury or disease, multiple sclerosis, spasticity, cerebral palsy, stroke, and the like. Preferably, the neuromuscular disease associated with increased muscle tone and/or involuntary movement is blepharospasm or torticollis. More preferably, the neuromuscular disease associated with increased muscle tone with involuntary movement is blepharospasm.

In one embodiment, the present invention provides methods for producing denervation in a subject suffering from blepharospasm comprising administering between 10-200 LD₅₀ units of a composition of the present invention, as described herein. In another embodiment, the present invention provides methods for producing denervation in a subject suffering from torticollis. Preferably, the effective amount of a composition of the present invention is between 10 and 3000 LD₅₀ units.

In another embodiment, the present invention provides a method of treating a condition selected from the group consisting of facial wrinkles, rhytides and cosmetic alteration of lip and brow, in a subject in need thereof, comprising administering an effective amount of a composition of the present invention, as disclosed herein. Preferably, the effective amount is between 2.5 and 400 LD₅₀ units.

In yet another embodiment, the present invention provides a method of treating human headache disorders in a subject in need thereof, comprising administering an effective amount of a composition of the present invention, as disclosed herein. Preferably, the effective amount is between 5 and 1000 LD₅₀ units.

In a further embodiment, the present invention provides a method of treating human migraine headache disorders in a subject in need thereof, comprising administering an effective amount of a composition of the present invention, as disclosed herein. Preferably, the effective amount is between 5 and 1,000 LD₅₀ units.

The present invention also provides a method of treating human inflammatory conditions in a subject in need thereof, comprising administering an effective amount of a composition of the present invention, as disclosed herein. Preferably, the effective amount is between 5 and 4,000 LD₅₀ units.

The present invention also provides a method of treating myopathic or neuropathic pain in a subject in need thereof, comprising administering an effective amount of a composition of the present invention, as disclosed herein. Preferably, the effective amount is between 5 and 4,000 LD₅₀ units.

The present invention also provides a method of treating back pain or arthritic pain in a subject in need thereof, comprising administering an effective amount of a composition of the present invention, as disclosed herein. Preferably, the effective amount is between 5 and 4,000 LD₅₀ units.

In yet another embodiment, the present invention provides a method of treating gastrointestinal spasm and strictures in a subject in need thereof, comprising administering an effective amount of a composition of the present invention, as disclosed herein. Preferably, the effective amount is between 5 and 4,000 LD₅₀ units.

The present invention provides a method of treating a hyperhyrosis syndrome in a subject in need thereof, comprising administering an effective amount of a composition of the present invention, as disclosed herein. Preferably, the effective amount is between 5 and 4,000 LD₅₀ units.

The present invention provides methods of treating depressive, anxiety and sleep disorders comprising the administration of pharmaceutical compositions comprising neurotoxins.

The present invention provides methods for treating depression comprising the steps of: a) identifying a subject with a depressive disorder or identifying a subject with one or more symptoms of a depressive disorder; and b) administering an effective amount of a composition comprising a botulinum toxin and a pharmaceutically acceptable carrier to said subject.

The present invention also provides methods of treating anxiety comprising the steps of: a) identifying a subject with an anxiety disorder or identifying a subject with at least one symptom of an anxiety disorder; and b) administering an effective amount of a composition comprising a botulinum toxin and a pharmaceutically acceptable carrier to said subject.

The present invention also provides methods of treating sleep disorders comprising the steps of: a) identifying a subject with a sleep disorder or identifying a subject exhibiting at least one symptom of a sleep disorder; and b) administering an effective amount of a composition comprising a botulinum toxin and a pharmaceutically acceptable carrier to said subject.

The present invention also provides methods of treating circadian rhythm disorders comprising the steps of: a) identifying a subject with a circadian rhythm disorder; and b) administering an effective amount of a composition comprising a botulinum toxin and a pharmaceutically acceptable carrier to said subject.

The present invention also provides methods of delivering botulinum toxin across a blood-brain barrier comprising the steps of: a) identifying a subject with at least one neuropsychiatric disorder; and b) administering a composition comprising a neurotoxin and a pharmaceutically acceptable carrier to said subject in an amount sufficient to deliver said neurotoxin across the blood-brain barrier.

The present invention also provides methods of delivering botulinum toxin across a blood-brain barrier comprising the steps of: a) identifying a subject with at least one neuropsychiatric disorder; and b) administering a composition comprising a neurotoxin and a pharmaceutically acceptable carrier to said subject in an amount sufficient to deliver said neurotoxin across the blood-brain barrier, wherein said administration of said injection of neurotoxin blocks at least one neurotransmitter. In a preferred embodiment, the neurotransmitter is acetylcholine.

The present invention also provides methods of treating an anxiety disorder comprising the steps of: a) identifying a subject with at least one anxiety disorder or identifying a subject with one or more symptoms of an anxiety disorder; and b) administering to said subject a composition comprising a neurotoxin and a pharmaceutically acceptable carrier said composition is delivered across the blood-brain barrier in an amount sufficient to decrease cholinergic neuron transmission.

The present invention also provides methods of treating a sleep disorder comprising the steps of: a) identifying a subject with at least one sleep disorder or identifying a subject with one or more symptoms of a sleep disorder; and b) administering to said subject a composition comprising a neurotoxin and a pharmaceutically acceptable carrier said composition is delivered across the blood-brain barrier in an amount sufficient to decrease cholinergic neuron transmission. In a preferred embodiment, the composition decreases choline acetyltransferase activity. In another preferred embodiment, the composition decreases the synthesis of acetylcholine. In another preferred embodiment, the sleep disorder is insomnia. In another preferred embodiment, the sleep disorder is narcolepsy, restless leg syndrome or sleep apnea.

The present invention also provides methods of treating a circadian rhythm disorder comprising the steps of: a) identifying a subject with at least one circadian rhythm disorder or identifying a subject with one or more symptoms of a circadian rhythm disorder; and b) administering to said subject a composition comprising a neurotoxin and a pharmaceutically acceptable carrier said composition is delivered across the blood-brain barrier in an amount sufficient to decrease cholinergic neuron transmission. In a preferred embodiment, the composition decreases choline acetyltransferase activity. In another preferred embodiment, the composition decreases the synthesis of acetylcholine.

The present invention also provides methods of treating a depressive disorder comprising the steps of: a) identifying a subject with at least one depressive disorder or identifying a subject with one or more symptoms of a depressive disorder; and b) administering to said subject a composition comprising a neurotoxin and a pharmaceutically acceptable carrier said composition is delivered across the blood-brain barrier in an amount sufficient to decrease cholinergic neuron transmission. In a preferred embodiment, the composition decreases choline acetyltransferase activity. In another preferred embodiment, the composition decreases the synthesis of acetylcholine.

In another embodiment of the present invention, subjects suffering from a condition selected from the group consisting of a depressive disorder, an anxiety disorder and a sleep disorder were identified by determining that a subject has a medical history of a depressive disorder, an anxiety disorder, or a sleep disorder, respectively.

The present invention provides methods for delivering a botulinum toxin based pharmaceutical to the central nervous system of a subject by any injection or topical application method, except intracranial, transcranial, intrathecal or intraspinal injection, in a therapeutically effective amount sufficient to decrease at least one central nervous system neurotransmitter when compared to an untreated subject. In a preferred embodiment, the at least one central nervous system neurotransmitter is glutamate, norepinephrine, or acetyl-choline. In a more preferred embodiment, the at least one central nervous system neurotransmitter is glutamate. In another embodiment, the methods of the present invention decrease at least one central nervous system neurotransmitter when compared to an untreated subject sufficiently to reduce at least one symptom of insomnia, a sleep disorder, an anxiety disorder, a depressive disorder, dysmenorrhea, an appetite or eating disorder, or a seizure disorder. In a preferred embodiment the seizure disorder is generalized, focal motor, or partial complex.

Glutamate is a neurotransmitter that exhibits endogenous neurotoxic activity that is observed in a number of neurodegenerative diseases and disorders, vascular accidents such as stroke and in seizure disorders. For example, subjects with mild to moderate dementia and probable Alzheimer's Disease have been shown to exhibit elevated levels of glutamate in the central nervous system. Elevated glutamate in the central nervous system is reflective of increased glutamatergic activity in the early stages of Alzheimer's Disease. The progressive neuronal loss observed in Alzheimer's Disease and other neurodegenerative disorders and diseases correlate with elevated glutamate and the increased excitotoxicity associated with elevated levels of this neurotransmitter.

The present invention provides methods for reducing glutamate levels in the central nervous system, the brain or portions of the brain comprising the step of administering a botulinum toxin pharmaceutical to a subject, by any injection or topical application method, except intracranial, transcranial, intrathecal or intraspinal injection, in an amount sufficient to reduce glutamate levels in the central nervous system, the brain or portions of the brain compared to an untreated subject.

The present invention provides methods for neuroprotection comprising the step of administering a botulinum toxin pharmaceutical to a subject, by any injection or topical application method, except intracranial, transcranial, intrathecal or intraspinal injection, in an amount sufficient to reduce neuronal loss in the central nervous system, the brain or portions of the brain compared to an untreated subject.

The present invention also provides methods for delivering a botulinum toxin based pharmaceutical to the central nervous system of a subject by injection into the nasal sinuses in a therapeutically effective amount sufficient to decrease at least one central nervous system neurotransmitter when compared to an untreated subject. In a preferred embodiment, the at least one central nervous system neurotransmitter is glutamate, norepinephrine, or acetyl-choline. In a more preferred embodiment, the at least one central nervous system neurotransmitter is glutamate. In another embodiment, the methods of the present invention decrease at least one central nervous system neurotransmitter when compared to an untreated subject sufficiently to reduce at least one symptom of insomnia, a sleep disorder, an anxiety disorder, a depressive disorder, dysmenorrhea, or a seizure disorder. In a preferred embodiment the seizure disorder is generalized, focal motor, or partial complex.

The present invention also provides methods for delivering a botulinum toxin based pharmaceutical to the central nervous system of a subject by any injection or topical application method, except intracranial, transcranial, intrathecal or intraspinal injection, in a therapeutically effective amount sufficient to decrease at least one central nervous system neurotransmitter when compared to an untreated subject. In a preferred embodiment, the at least one central nervous system neurotransmitter is glutamate, nor-epinephrine, or acetyl-choline. In a more preferred embodiment, the at least one central nervous system neurotransmitter is glutamate. In another embodiment, the methods of the present invention decrease at least one central nervous system neurotransmitter when compared to an untreated subject sufficiently to reduce an agitated behavior associated with mental retardation, schizophrenia, Huntington's Chorea or Alzheimer's Disease.

The present invention also provides methods for delivering a botulinum toxin based pharmaceutical to the central nervous system of a subject by any injection or topical application method, except intracranial, transcranial, intrathecal or intraspinal injection, in a therapeutically effective amount sufficient to decrease at least one central nervous system neurotransmitter when compared to an untreated subject. In a preferred embodiment, the at least one central nervous system neurotransmitter is glutamate, nor-epinephrine, or acetyl-choline. In a more preferred embodiment, the at least one central nervous system neurotransmitter is glutamate. In another embodiment, the methods of the present invention decrease at least one central nervous system neurotransmitter when compared to an untreated subject sufficiently to reduce at least one symptom of a neurodegenerative disease associated with inflammation.

The present invention also provides for the use of botulinum toxin or a botulinum toxin composition of the present invention in the production of a medicament for the treatment of any one of the disorders, diseases or conditions disclosed herein, including depressive disorders, anxiety disorders, sleep disorders, circadian rhythm disorders, neuropsychiatric disorders, Alzheimer's Disease and the like, and for the treatment of pain, such as various headache pain, associated with a pain syndrome.

The present invention also provides a method of producing the compositions described herein. In one embodiment, the method comprises mixing a hyaluronidase with botulinum toxin. In another embodiment, the method comprises freeze drying or flash drying a hyaluronidase with botulinum toxin.

### DETAILED DESCRIPTION OF THE INVENTION

The present invention describes a method and composition to enhance the clinical effectiveness of botulinum toxin preparation for clinical use by means of increasing the stabilization of the botulinum toxin composition. The present invention also describes a method and composition to enhance the delivery systems of pharmaceutical botulinum toxin compositions. Improved delivery systems can be useful to provide a molecular anchor to neurotoxin molecules preventing diffusion away from the injection point, causing maximal saturation of botulinum neurotoxin receptors, thereby achieving greater efficacy with the amount of neurotoxin used to achieve desired clinical effects.

### A. Definitions.

As used herein, “Botulinum toxin” means a protein toxin isolated from strains of Clostridium botulinum, including mixtures of its protein complexes, toxoid and/or other clostridial proteins. “Botulinum toxin” includes all of the various immunotypes such as A, B, C₁, C₂, C₃, D, E, F and G.

As used herein, “administration” of a composition means any route of administration, including but not limited to oral, nasal, transcutaneous, percutaneous, subcutaneous, intraperitoneal, transdermal, intramuscular and intraosseous, but expressly excludes intracranial, transcranial, intrathecal or intraspinal injection.

As used herein, “depressive disorder” means major depression, dysthymia, and atypical depression or depression not otherwise specified.

As used herein, “an effective amount” is an amount sufficient to produce a therapeutic response. An effective amount may be determined with dose escalation studies in open-labeled clinical trials or bin studies with blinded trials.

As used herein “neuromuscular diseases” refer to any disease adversely affecting both nervous elements (brain, spinal cord, peripheral nerve) or muscle (striated or smooth muscle), including but not limited to involuntary movement disorders, dystonias, spinal cord injury or disease, multiple sclerosis, and spasticity from cerebral palsy, stroke, or other cause.

As used herein, the term “pharmaceutically acceptable carrier” means a chemical composition, compound, or solvent with which an active ingredient may be combined and which, following the combination, can be used to administer the active ingredient to a subject. As used herein, “pharmaceutically acceptable carrier” includes, but is not limited to, one or more of the following: excipients; surface active agents; dispersing agents; inert diluents; granulating and disintegrating agents; binding agents; lubricating agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; antioxidants; stabilizing agents; and pharmaceutically acceptable polymeric or hydrophobic materials and other ingredients known in the art and described, for example in Genaro, ed., 1985, Remington's Pharmaceutical Sciences, Mack Publishing Co., Easton, Pa., which is incorporated herein by reference.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, and other mammals.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient. In addition to the active ingredient, a pharmaceutical composition of the invention may further comprise one or more additional pharmaceutically active agents. Particularly contemplated additional agents include anti-emetics and scavengers such as cyanide and cyanate scavengers. Controlled- or sustained-release formulations of a pharmaceutical composition of the invention may be made using conventional technology.

As used herein, the term “hyaluronidase” means any protein that catalyzes the hydrolysis of hyaluronic acid.

### B. Botulinum Toxin.

Botulinum toxin type A is the most lethal natural biological agent known to man. Seven immunologically distinct botulinum neurotoxin serotypes have been characterized—A, B, C₁, D, E, F and G. Each botulinum toxin serotype is distinguished by neutralization with type-specific antibodies. The different serotypes of botulinum toxin vary in the animal species that they affect and in the severity and duration of the paralysis they evoke.

Since its introduction as a therapeutic agent, the pharmaceutical measurement of the denervating or biologic activity of botulinum toxin has been the LD₅₀ Unit (LD₅₀ Unit and Unit are used interchangeably herein) determined by using 18-22 gram Swiss-Webster mice, quantitated statistically by injecting cohorts of mice at different dilutions from the purified botulinum neurotoxin protein and its protein complexes. This measurement has the advantage of a clear endpoint (living or dead mouse), however the LD₅₀ unit does not predict clinical behavior of various botulinum toxin formulations when compared in clinical studies.

Toxins of the different C. botulinum serotypes are produced in culture as aggregates of neurotoxin and other non-toxic proteins non-covalently associated into a polypeptide complex. (Schantz (1964) Purification and characterization of C. botulinum toxins, In Botulism. Proceedings of a symposium. K. Lewis and K. Cassel, Jr. (eds.), U.S. Department of Health, Education, and Welfare, Public Health Service, Cincinnati, pp. 91-104; Sugii et al. (1975) Infect. Immun. 12: 1262-1270; Kozaki et al., (1974) Jpn. J. Med. Sci. Biol. 28: 70-72; Miyazaki et al. (1977) Infect. Immun. 17: 395-401; Kitamura et al. (1969) J. Bacteriol. 98: 1173-1178; Ohishi et al. (1974) Appl. Environ. Microbiol. 28: 923-928; Yang et al. (1975) Appl. Microbiol. 29: 598-603). Toxin complexes are described as M for medium, L for large and LL for very large. These toxin complexes vary in size from about 900 kD for type A LL toxin complex to about 300 kD for the type B M complex and type E complex, to 235 kD for type F M complex. The Hall strain of type A Clostridium botulinum is preferably used for the production of type A neurotoxin. (Goodnough et al. (1992) Appl. Environ. Microbiol. 58(10): 3426-3428); Goodnough and Johnson (1994) ACS Symposium Series No. 567, J. Cleland and R. Langer (eds); Tse et al. (1982) Eur. J. Biochem. 122: 493-500). Botulinum neurotoxin may be prepared by culturing Clostridium botulinum, harvesting, solubilizing and purifying using standardized methods that ensure quality and sterility. (Schantz and Johnson (1992) Microbiol. Rev. 56: 80-99; (Goodnough et al. (1992) Appl. Environ. Microbiol. 58(10): 3426-3428); Goodnough and Johnson (1994) ACS Symposium Series No. 567, J. Cleland and R. Langer (eds); Tse et al. (1982) Eur. J. Biochem. 122: 493-500). incorporated herein by reference in its entirety).

### C. Potency

The potency of a particular botulinum toxin preparation or formulation may be determined clinically or in animal models of muscle denervation. Clinically, a first botulinum toxin preparation exhibits greater potency than a second preparation when fewer LD₅₀ Units of the first preparation are required to achieve a desired therapeutic effect.

In animal models, the potency of a botulinum toxin preparation may be determined by measuring the extent of denervation produced when a preparation is administered to a muscle. Post mortem sectioning of rabbit muscle about a site of toxin injection, demonstrates that botulinum toxin produces a gradient of denervation similar to that observed in mouse muscle (Duchen (1970) J. Neurol. Neurosurg. 33:40-54; J. Physiol. (Lond) (1969) 204:17-18). The extent of this denervation gradient (a measure of the spread of a given dose of the toxin) is a measure of potency. Animal models for muscle denervation are disclosed and described in U.S. Pat. No. 5,298,019, which is incorporated herein by reference in its entirety.

The longissimus dorsi muscle of New Zealand white rabbits is the preferred animal model for determining the denervating potency of a botulinum toxin preparation. Denervation may be assessed by any available analytical method. For example, denervation may be determined at various distances from the injection site by post mortem sectioning of the treated muscle and staining for acetylcholinesterase activity. Techniques for acetylcholinesterase-activity staining are described by Karnovsky (See Woolf and Coers, The Innervation of Muscle, Charles Thomas Pub, Springfield, Ill., 1959, which is incorporated by reference herein in its entirety). Inhibition of acetylcholine release may also be measured by single-fiber electromyography (See, for example, Sanders et al. (1985) Botulinum Toxin for Blepharospasm, Single Fiber EMG Studies, Neurology 35: 271-272). Labeled binding proteins, including polyclonal or monoclonal antibodies, may also be used to detect acetylcholinesterase, acetylcholine receptors, and acetylcholinesterase activity. Binding proteins may be labeled using, for example, fluorescein or other fluorescent moieties, colloidal metallic particles, other remotely-detectable substances, and the like. Antibodies can be produced, using known techniques, to acetylcholine receptors or to acetylcholinesterase, both of which can serve as a marker for effective denervation, or to epitopes which are newly exposed, or which remain after binding of the toxin to the receptor on the presynaptic motor end plate. Other stains such as hematoxylin, eosin, masson trichrome, and the like may also be used.

### D. Hyaluronidase

Recent advances in pharmaceutical technology have focused on enhanced delivery systems such as transdermal or transcutaneous delivery systems. Such systems are thought to be more convenient and associated with less pain. The problems associated with such systems include poor penetration of materials through the epidermis and dermis. The hyaluronidase offers improved penetration by causing dispersion through the ground substance of connective tissues.

Herein is described a combination of botulinum neurotoxin, hyaluronidase, and sugars (both simple and oligosaccharides) for therapeutic injection. The formulation will be devoid of any human blood or recombinant blood products and will be either stabilized in flash or freeze dried form. The pH will be from 3.0 to 7.4 and the preparation may be used as an injection, transdermal or topical agent. The combined pharmaceutical can be administered by injection, needleless delivery systems and methods requiring disruption techniques such as electroporation, sonication, and high pressure air gas flow injection or in the form of a micro-needle. Micro needles are generally from 150 to 600 microns. Furthermore, the composition may further comprise polycationic proteins.

Prior studies have show that a protein excipient, such as human serum albumin, can stabilize botulinum toxin formulations. Test studies demonstrate that hyaluronidase also stabilizes botulinum toxin formulations at the same or greater levels observed for human serum albinum. In one test study, two compositions of botulinum toxin, sugar, and hyaluronidase were prepared at 4° C. and at 37° C. One composition contained a concentration of 0.1 mg/mL hyaluronidase; and a second composition contained 0.5 mg/mL hyaluronidase. The compositions were administered at doses of 1, 10, 50, and 100 mouse LD₅₀ units of botulinum toxin. The compositions were injected into two mice at three different times: (1) immediately following the preparation of the composition; (2) seven days after preparation of the composition; and (3) fourteen days after preparation of the composition. Two similar compositions were prepared containing human serum albumin in the same concentration as, and instead of, the hyaluronidase. The human serum albumin compositions were administered to two mice in the same manner as the hyaluronidase compositions were administered.

At 4° C., and at a concentration level of 0.1 mg/mL, the hyaluronidase showed a stabilizing effect similar to the human serum albumin at the same concentration level. At 4° C., the 0.5 mg/mL hyaluronidase composition produced similar stabilizing results to the composition with 0.5 mg/mL human serum albumin. Similar results were demonstrated at all dose levels.

At 37° C. and at a concentration level of 0.1 mg/mL hyaluronidase, the hyaluronidase composition produced results similar to the composition containing 0.1 mg/mL human serum albumin. At 37° C., the composition containing 0.5 mg/mL of hyaluronidase was as stable or more stable than the composition containing 0.5 mg/mL human serum albumin. Following injection of the hyaluronidase composition after seven and fourteen days of storage at 37° C., the test subjects remained alive after injections of 10, 50, and 100 mouse LD₅₀ units of botulinum toxin.

One disadvantage of using recombinant human serum albinum in botulinum toxin compositions is the presence of immunogens which cause local inflammation after repeated injections. Botulinum-based pharmaceutical preparations require repeated injections, usually at three to four month intervals. The human serum albumin composition must be qualified with respect to a local allergy at a rate of less than 1% for the composition to meet a minimally acceptable standard. Any local or systematic response to the recombinant serum albumin would make the use of the botulinum toxin unfeasible, with an adverse risk/benefit ratio for the patient.

An advantage of the compositions described herein is that hyaluronidase reduces antigenic response to botulinum toxin. In a preferred embodiment, the hyaluronidase is present in an amount sufficient to induce substantially no antigenic response to botulinum toxin. In another embodiment, the hyaluronidase is present in an amount sufficient to induce substantially no allergic reaction, local inflammation, and/or local sensitivity or other irritation.

### E. Neuromuscular Disorders

The botulinum toxin formulations of the present invention may be used to treat a variety of neuromuscular disorders that are characterized by involuntary muscle contractions and/or spasms. These neuromuscular disorders include, but are not limited to dystonias, including cervical dystonia (spasmodic torticollis), spasmodic dysphonia, hemifacial spasm, blepharospasm, bruxism, and spasticity caused by cerebral palsy, stroke, and the like.

Cervical dystonia or spasmodic torticollis is a focal dystonia characterized by neck muscles contracting involuntarily, causing abnormal movements and posture of the head and neck. The abnormal movements and spasms may occur in any direction. Contractions producing forward movements are frequently referred to as anterocollis, whereas spasm that produce backwards or sideways movements are referred to as retrocollis and torticollis, respectively. The movements may be sustained or sporadic. Sustained contractions produce abnormal head and neck posture, whereas periodic spasm produce jerky head movements. The spasms and muscle contractions that produce cervical dystonia are also associated with considerable neck pain and discomfort.

The cause of cervical dystonia is unknown, but is believed to be associated with defects in the basal ganglia which control movement. Although a dopamine deficiency or imbalance may be the underlying chemical basis for the disorder, the exact etiology of cervical dystonia remains unknown. Cervical dystonia may be diagnosed through a medical history, physical and neurological examination. Currently, there is no laboratory or clinical test to confirm a diagnosis of blepharospasm.

Cervical dystonias usually increase in severity, reaching a plateau and remaining stable within five years after onset. This form is unlikely to spread or become generalized dystonia, though patients with generalized dystonia may also have cervical dystonia. Occasionally, there may be associated focal dystonia. Cervical dystonia should not be confused with other conditions which cause a twisted neck such as local orthopedic, congenital problems of the neck, ophthalmologic conditions where the head tilts to compensate for double vision. It is sometimes misdiagnosed as stiff neck, arthritis, or wry neck.

Botulinum toxin injections are the primary and most effective form of treatment for cervical dystonia. Injections are made directly into the affected neck muscles. A crucial element to successful botulinum toxin injections is that the appropriate muscles are injected. For example, the muscles most commonly involved in cervical dystonia include the splenius capitis, the levator scapulae, upper trapezius, sternocleidomastoid, anterior, middle and posterior scalene. The Dystonia Medical Research Foundation, for example, recommends low-dose (about 150 U BOTOX®) administration of botulinum toxin to avoid immunity. Single and especially chronic dosing with greater than 200 U BOTOX® greatly increases the risk of inducing the production of neutralizing antibodies and resistance to the toxin.

### F. Neurosensory Disorders (Pain)

The botulinum toxin formulations of the present invention may also be used to treat a variety of sensory disorders such as pain syndromes, including myofascial pain, migraine, tension headaches, post-operative wound pain, nerve compression, neuralgias, trigeminal neuralgia, pain associated with cervical dystonia and other dystonias, neuropathy, and sinusitis-related facial pain.

Sinus-related headaches are distinctly different from migraine headache, myofascial headaches, and headaches associated with bruxism, temporal mandibular joint syndrome (TMJ) and temporal mandibular muscle dysfunction (TMD), trigeminal neuralgia, tooth related facial pain, pain associated with elevated intraocular pressure, or internal ocular inflammation. Sinus headaches are associated with pressure, or irritating processes within the sinus cavities, sometimes associated with inflammation and impaired flow of mucous secretion. At some point in the diagnostic workup, excessive signs of inflammation within the sinus or nasal cavity, or edema within the sinus or nasal cavity is demonstrated on exam or via radiographic methods. The present inventors have discovered that botulinum toxin relieves the headache and facial pain associated with sinusitis.

The present invention provides methods of treating headache and facial pain associated with acute recurrent or chronic sinusitis in a subject in need thereof, comprising the step of administering a therapeutically effective amount of a composition comprising botulinum toxin to the nasal mucosa or to the subcutaneous structures overlying the sinuses, wherein the administration of the composition reduces the headache and facial pain associated with acute recurrent or chronic sinusitis. In a preferred embodiment, the sinuses are one or more of the sinuses selected from the group consisting of: ethmoid; maxillary; mastoid; frontal; and sphenoid. Preferably, the subcutaneous structures overlying the sinuses lie within one or more of the areas selected from the group consisting of: forehead; malar; temporal; post auricular; and lip.

Botulinum toxin may be administered to the nasal mucosa or to the subcutaneous structures overlying the sinuses by any number of methods. Preferably, the composition comprising botulinum toxin is administered by injection at one or more injection sites. More preferably, the composition comprising botulinum toxin is administered to the cutaneous projections of the trigeminal nerve innervating the sinus.

In one embodiment of the present invention, a subject is treated by administration of a composition comprising botulinum toxin, wherein the subject, prior to the onset of facial pain or headache, exhibits symptoms or history of sinus rhinorrhea (nasal hypersecretion) and purulent nasal discharge.

Sinusitis is defined as any inflammatory pathology involving the ethmoid, maxillary, frontal, or sphenoid sinuses. It is generally accepted that the cause of pain occurring with acute sinusitis involves infiltration of sinus mucosa with inflammatory cells, as well as increased pressure within the sinuses. What is generally not appreciated, and is herein disclosed, is that sinusitis can cause sensitization of the trigeminal nerve in cutaneous and subcutaneous tissues overlaying the sinus structures. When sensitization of sensory nerves occurs from repeated bouts of sinusitis, the patient can experience a chronic facial pain syndrome or headache. The mechanism by which sensory nerves become up-regulated or sensitized still is not clear. Nerve sensitization is provoked by alterations in the afferent first-order-sensory nervous system, such that thresholds are lowered to the perception of pain (hyperalgesia) and central second-order or higher-neuronal alterations can occur, resulting in an exaggerated response and interpretation of sensory stimuli (central sensitization). This process has been experimentally associated with increased expression and/or responsiveness of NMDA receptors on membranes of nociceptors and possible alterations in transcription and translation of proteins within the nerve cell. The trigeminal ganglia represent a very large collection of afferent sensory neurons, which send projects not only into cutaneous regions of the head, but also internally into osseous sinus structures, and mucous membranes of the nasal and sinus cavities.

The arborization pattern of afferent sensory nerve distribution is extensive, but reactivity within any region of the afferent sensory nerve distribution has the capability of altering the genetic and cellular-protein expression of the sensory nerve cell body within the ganglion. The process of changing cell physiology has been variously coined neuroplasticity or sensitization. Alterations can be in the form of increased expression of nerve cell receptors, such as AMPA and NMDA receptors, modulation of effectors of inflammation, alteration of cellular responses from blood-vessel neural regulation via nitric oxide, substance P, histamine, CRGP, prostaglandins, other known cellular autocoids, and not yet defined autocoids and neuropeptides. The mechanism for sensitization of human nerve cells is still not well understood, and invoking inflammatory mediators, neurogenic inflammatory autocoids, and transcriptional and phenotypic changes of nociceptors and sensory neurons as the only mechanisms for nerve sensitization is not necessary to elicit responses from therapeutic botulinum toxin for this indication. Sensitization in the periphery is thought to occur following a sufficient or prolonged exposure to inflammatory substances, causing altered physiology, possible conformational changes of certain biochemical receptors, responsiveness, and lowered thresholds for nociceptor and sensory nerve depolarization.

Sinus pain usually begins in the mid facial region over the maxillary sinus and can radiate to temporal regions, ocular regions, vertex, and over the forehead. At times, referred pain can project into the posterior cervical region or peri-auricular areas. Generalized headaches can occur. The trigeminal nucleus is somatotropically well organized, and from the brain stem area, directly extends and connects anatomically to the upper-cervical areas of the dorsal horn of the spinal cord. In addition, there are interneuronal connections between the trigeminal nucleus and other cranial nerve nuclei, the autonomic nervous system, the reticular activating system, and other descending and ascending pathways. This interconnecting system has been described as the trigeminal sensory complex. Since there are many more peripheral upper cervical and trigeminal sensory nerves synapsing on fewer central nerves, this has been described as convergence and projection. This can explain the referral patterns of head and neck pains, and the therapies employed in one area of the head and neck to affect an outcome on a another area of the head and neck with shared and referred sensory pathways.

Distinct differences in headache diagnosis have been formulated at international conventions and remain the basis for both general and research practice. For migraine headaches, the presence of episodic headaches lasting 4-48 hrs, associated with light sensitivity (photophobia), sound sensitivity (phonophobia), nausea or vomiting, pain of a throbbing or pulsating quality, and more often unilateral than bilateral location of headache. Cluster headaches can be associated with some basal transient nasal congestion but occur over a distinct time period (cluster period) and are not associated with any persistent sinus abnormalities on MRI or computerized tomography. Myofascial and tension headaches often have a cap-like squeezing pain across and around the top of the head, often associated with a cervical musculoskeletal pain location, frequently associated with trigger points, and sometimes associated with decreased jaw motility and bruxism if the masseter and temporalis muscles are involved. Ocular-related headaches are associated with increased intra-ocular pressure or signs of intra-ocular inflammation on slit lamp microscopic exam or measured refractive error. Dental-related headaches are associated with findings on dental examination and radiographs. Trigeminal neuralgia is usually limited to one or two dermatomes and is sharp and stabbing in quality, with a rapid “on-off” episodic pattern sometimes associated with stimulation of trigger points.

Chronic-sinusitis-related headache and facial pain can linger for many months to years after an acute or subacute bout of sinus disease or bout of repeated acute sinus headaches. Often, the patient complains of continued pain when radiologic imaging studies, such as computerized tomography and magnetic resonance imaging fail to show any persisting signs of inflammation such as mucosal thickening or fluid accumulation. Often out of desperation, the surgeon performs decompressive surgery via endoscopes or direct approaches (Culdwell luc, external ethmoidectomy) with poor results with respect to the chronic pain. The above observation explains a very common clinical phenomenon associated with chronic facial pain and headache caused by sinusitis. The reason for the persisting pain despite the absence of active sinus findings is peripheral sensory nerve upregulation or sensitization. Direct treatment of sinus-related headache by botulinum toxin injected into the subcutaneous region to down-regulate sensory nerves is therapeutic.

The convention in treating sinus-related headaches involves decongestants to augment mucous clearance and drainage from sinus cavities, antibiotics to treat bacterial infection, anti-inflammatory medication (e.g. corticosteroids), and surgical decompression. Conventional analgesics such as aspirin and acetaminophen may be used. The present inventors have made the unexpected discovery that administration of botulinum toxin over the surface dermatomes containing the sensory branches corresponding to the neurons projecting into the sinus cavity effectively treats facial and headache pain associated with sinusitis.

A convention held in 1985 by the International Headache Society (I.H.S.) put forth an exhaustive classification of distinct headache syndromes. Experts in the headache therapeutic field formulated this classification, and such experts explicitly agreed on the importance of headache distinction both for practice and research. The reasons for distinctions are to promote better communication among practitioners and to provide more exacting therapy for specific headache syndromes. For instance, procedures used to treat trigeminal neuralgia, such as glycerol injections, gamma knife application, and microvascular decompression at the level of the brainstem are not effective for the treatment of recurrent sinus headache. Tryptin-related pharmaceuticals (e.g. Imitrex™, Zomig™)) would be ineffective for the treatment of sinus headache and laser iridectomy for the treatment of narrow angle glaucoma would be ineffective for the treatment of migraine. Cluster headache needs to be distinguished from migraine. Hence, one skilled in the art of treatment of pain would require specific and professionally acceptable diagnosis in order to recommend reasonable therapy or to conduct clinical trials with potentially effective new therapies. The convention held in 1985 and subsequently published in Cephalgia (1988 Vol 8 (supplement 7), 1-96) has served as a benchmark for diagnosis and classification of human headaches (nosology) for the past 15 years.

In order for the physician to function and recommend therapeutic interaction with patients suffering from pain, classification with diagnostic criteria of an affliction must be determined. Classification of disease must be operationally specified with quantitative parameters and not just descriptive. The International Headache Society (I.H.S.) formed a committee in 1995 which lead to the first adopted international headache classification, which in turn permitted uniform operational criteria for diagnosis. The I.H.S. is internationally accepted and has been incorporated into the World Health Organization (W.H.O.) classification of disease. This classification has been translated into multiple languages and competes with no other classification system (see Jes Olesen Classification of Headache in Chapter 2, The Headaches, 2^{nd} Edition, Lippincott, Williams and Wilkins ed Olesen, Hansen, Walsh, Philadelphia, 1999).

In the classification system, headaches in category 1-4 are primary headache disorders with no associated anatomic pathologic process. Groups 5-11 are headaches and cervical pain associated with some other demonstrable disease process (trauma, vascular disease, increased intracranial pressure, withdrawal from substances, systemic infection, metabolic disorder, eye, ear, nose, and throat disease, or dental disease. Group 12 relates to cranial neuralgias.

### H. Inflammation

Inflammation is a normal response to tissue damage. Inflammation is often characterized by edema, erythema and pain. Acute inflammation may caused by a variety of injury, including physical and chemical injury and tissue damage caused by microorganisms and other agents. The inflammatory response consist of changes in blood flow, increased permeability of blood vessels and the escape of cells from the blood into the tissues.

Acute inflammation is short-lasting, lasting only a few days. Chronic inflammation is characterized by a longer duration. Examples of acute inflammation include hives, swelling, itching and pain associated with insect bites, burns or exposure to a chemical agent or allergen. Inflammatory conditions may also affect internal organs such as the lungs, gastro-intestinal tract, heart, kidneys and the like.

Disease known to be inflammation driven in etiology include rheumatoid arthritis, inflammatory bowel disease, Crohn's Disease, interstitial cystitis, eczema, hay fever, inclusion arthritis, myositis, post surgical inflammatory states, reflex sympathetic dystrophy, arteritis, nephritis, scleroderma, asthma, prostatitis, sarcoidosis, bacterial infections, seborrhea, acne, osteomyleotitis, wound healing sites, systemic lupus erythematosis, Stevens Johnson syndrome, cutaneous and deep burns, myofascial pain syndromes, osteoarthritis, conjunctitis, blepharitis, uveitis, sialoadenitis, gastritis, tendonitis, keratitis, and post traumatic tissue damage, and the like.

Botulinum toxin in doses lower than that necessary to treat regional movement disorders has been shown to reduce inflammation and adverse sensory experiences associated with the inflammatory response. These observations are explained by the fact that it has been found that low dosages of the subject chemodenervative agent reduces histamine releases and releases of other preformed mediators associated with mast cell degranulation. The anti-inflammatory activity is observed at low doses in animal models for ocular surface disease that are well noted for histamine release and release of other preformed mediators associated with mast cell degranulation and rapid inflammatory response. Accordingly, botulinum toxin blocks edema, erythema, abnormal sensory experiences, and heat transfer that occur rapidly over a predefined region.

The anti-inflammatory action of botulinum toxin is explained by the resultant blockage of mast and nerve cell release of histamine and other preformed mediators which result in vascular dilation, increased permeability, altered sensory experience, edema and erythema—the hallmarks of the rapid-phase inflammatory response. It will be appreciated that mast cells are known to contain a number of substances important to inflammatory responses in hypersensitivity reactions, and substantially participate in more generalized inflammatory reactions. The mast cell is abundantly found in pathologic tissue specimens in patients with rheumatoid arthritis, inflammatory bowel disease, certain forms of ocular uveitis, eczema, and asthma.

Mast cell activation has been associated with the production of both preformed mediators such as histamine, newly formed mediators such as leukotrienes and prostaglandins, cytokines, including interleukin-5, interleukin-8, kininogenase, and platelet activating factor. A number of these mast cell constituents play a role in the inflammatory response functioning as chemoattractants, activators and spasmogens. Additionally, a number of these constituents are activated and released in response to neural stimulation and play a role in neural sensory adaptation systems. Histamine is well known to produce itching sensation causing a compulsion to scratch or stimulate the activated area. Histamine also causes pain in patients with genetic predisposition to develop essential headaches.

An especially important cytokine identified as being important to inflammation and pain is tumor necrosis factor alpha. Tumor necrosis factor alpha has been identified in activated mast cells, and plays a role in modulation of mast cell activity. (Cocchiara et al. (1999) Histamine and Tumor Necrosis Factor-alpha Production from Purified Rat Brain Mast Cells Mediated by Substance P. Neuroreport 10(3):575-8; Olejnik et al. (1998) Tumor Necrosis Factor Alpha (TNF-alpha) Modulates Rat Mast Cell Reactivity. Immunol. Lett. (2-3): 167-71). Anti-tumor necrosis factor, as well as other pre-formed and newly formed mediators are autocoids which are reduced when suppressing mast-cell releases.

The botulinum toxin formulations of the instant invention are given in a therapeutically effective dose to reduce inflammation, and may be used in any application in which inflammation is present or to augment other inflammatory agents. The administration may be by injection, topical application, or other means to assure a therapeutically effective dose delivered to the site. Not only is the subject treatment efficacious in disease treatment normally associated with the occurrence of inflammation, it is also efficacious in the treatment of other diseases. Note that mechanical or adjuvant chemical activity may be necessary to increase penetration by topical application.

Urticaria refers to the formation of hives occurring usually in response to allergic reactions to pollens, foods, dander or other forms of antigens. The process often involves binding of allergens to the IgE receptor of the mast cell membrane bound IgE, causing release of preformed mediators such as histamine and serotonin as well as newly formed mediators from arachadonic acid such as prostaglandins and leukotrienes, platelet activating factor, kinoginase and tryptase, as well as cytokines. A late response can be seen after an allergic urticaria reaction which may be painful.

Urticaria may be provoked by non-allergens, including codeine, morphine, compound 48/80, synthetic ACTH, and anaphylatoxins C3a, C5a. Important, relative to the case observation, is the reactivity of mast cells to acetylcholine. (Fantozzi et al. (1978) Release of Histamine from Rat Mass Cells by Acetycholine. Nature 273 (5662): 473-4).

Mast cells are known to be abundant around blood vessels in the scalp, orbit and lids, and are thought to be important in allergic conjunctivitis. (Allensmith et al. (1981) Percentage of Degranulated Mast Cells in Vernal and Giant Papillary Conjunctivitis. Am. J Ophthalmol. 9: 71-75; Henriquez et al. (1981) Mast Cell Ultrastructure, Comparison in Contact Lens-associates Giant Papillary Conjunctivitis and Vernal Conjunctivitis. Arch. Ophthalmol. 99: 1266-1272). Mast cell reactivity has been associated with hayfever blepharoconjunctivitis, asthma, allergic rhinitis, and allergic forms of eczema. Mast cells are also seen abundantly in inflammatory responses in rheumatoid arthritis and inflammatory bowel disease.

Mast cells are closely associated with Type-1 hypersensitivity reactions. In such reactions, the typical response involves sensitization with an antigen, formation of immunoglobulin, IgE class, binding of immunoglobulin to the external cell membrane by its FcE receptor, and setting the stage for hypersensitivity to the second exposure to the antigen. Upon second exposure, IgE reacts with the antigen effect in a degranulation response of the mast cell, in which there is a release of preformed mediators such as histamine and serotonin, platelet activating factor, and newly formed mediators such as leukotrienes, prostaglandins, tryptase, kininogenase which effect vasodilatation, vascular permeability, micro thrombi, edema, mucous secretion. The response persists manifesting a late response after 8 hours. The late response is associated with pain as described by Roit, I., Brostoff, J., Male, D., Immunology 5^{th} Edition Mosby, 1998.

Internal inflammatory diseases may also be treated with botulinum toxin. In the past, it was thought that the tissue mechanisms associated with using chemodenervating agents have solely involved the use of botulinum toxin as a means of causing muscle relaxation or to produce certain autonomic effects blocking decreased sweating. Although there have been conditions treated by chemodenervating agents which have had associated inflammatory reaction as a part of the clinical syndrome, the concept of muscle relaxation induced by such agents has been thought to be the mechanism by which such agents induce the beneficial effects. It has now been found that the subject agent has useful anti-inflammatory properties capable of blocking ocular surface allergic inflammation in man and animal models, as well as generalized inflammation within the denervation field created.

For treatment, the practitioner defines a fixed anatomic area in which symptomatic and/or destructive inflammatory processes are occurring. Knowledgeable of dose related diffusion properties and potency of the preparation being used, the practitioner defines the anatomic area to be treated. Avoiding critical structures, e.g. blood vessels, nerves and anatomic cavities, the practitioner injects a fixed dosage of the chemodenervating agent so as to create a denervation field reducing the intensity of tissue destruction occurring within the area of treatment. Such a field can be defined internally, e.g. stomach mucosae-gastrits, joint-arthritis and muscle myositis. Follow-up involves monitoring for the cardinal sign of inflammation-pain redness, edema and discharge. Adjuvant therapy with other anti-inflammatory agents would be contemplated.

One of the most devastating chronic internal inflammatory diseases is rheumatoid arthritis, characterized by joint and periocular involvement and chronic inflammatory causing destruction of cartilage and ligamentous structures involving joints throughout the body. Immunologic causes have been cited as the underlying pathologic mechanism of the chronic destructive process, and mast cells have been noted in large quantities within the tissue pannus surrounding joints afflicted. Edema, joint effusions, stiffness, spasms, pain, and erythema, are all components of the arthritis involved regions. Multiple anti-inflammatory agents have been tried, with variable results to suppress the destructive effects of this systemic disease on bone and joints.

The formulations of the instant invention offer a means of localized application of an anti-inflammatory agent which is injected directly into joints or perarticular muscular tissues which creates an effect on the rapid inflammatory response and peripheral neural elements governing the inflammatory response. The application may be repeated at 3-month intervals and at titrated doses by clinical methods so as to limit any weakness within the injected region.

### I. Cosmetic Applications

Lines and wrinkles of the skin are the products of multiple causes that reduce the collagen and fat content of the skin, including aging and sun exposure. Aging produces wrinkles that may be characterized as fine lines that disappear when the skin is stretched. Wrinkles and lines resulting from sun damage are coarser and deeper and do not disappear when the skin is stretched. The treatment for wrinkles varies with the degree of severity.

In some cosmetic applications, the botulinum toxin formulations of the present invention may be administered to the muscles of the face, including the forehead and eye area, to reduce lines and wrinkles. The disclosed botulinum toxin formulations may be administered through a variety of modalities including surface application, subcutaneous and intramuscular injection. Specifically, botulinum toxin may be used, for example, to treat glabellar frown lines, crow's feet, horizontal forehead lines, nasolabial fold, mental crease, upper lip, platysmal bands, horizontal neck lines and wrinkles of the lower part of the face. Generally, one to five injections are given per muscle. The selection of muscles and the number of injections per muscle, however, depend on the desired effect and the severity of the lines and wrinkles and are within the skill of the treating physician. Administration of botulinum toxin produces smoothing of the skin and reduction of fine lines and superficial wrinkles in the area of treatment.

The botulinum toxin formulations of the present invention are particularly suitable for use in methods for cosmetically modifying soft-tissue features. In particular, these soft tissue features are features of the face and neck. For example, the disclosed formulations may be used to alter the shape and volume of facial features such as the lips. Hypervolemic lips, for example, are anatomically caused by one or more of the following structural deviations: 1) excessive tone of lip retractor function of the certain facial muscles such as levator labii superioris, zygomaticus major and minor, levator labii inferioris, platysma, and depressor labii inferioris; excessive prominence and development of orbicularis oris muscle; and excessive non-muscular soft tissue volume within the lip itself. As a consequence of the long duration of botulinum-toxin-induced neuromuscular blockade, catabolism occurs within the innervated, striated muscle that produces shrinkage of muscle fiber and decreased muscle bulk and size. Consequently, administration of the disclosed formulations to muscles of the lip provide a method to reduce the shape and volume of the lips. The formulations disclosed herein, may be injected at one or more locations and muscles to produce cosmetic modification of the soft tissue in the area of administration. Multiple administration of botulinum toxin may be required to achieve the desired degree of muscle shrinkage and the cosmetic modification of soft tissues.

### J. Cutaneous Disorders

The botulinum toxin formulations of the present invention may also be used to treat a variety of cutaneous disorders, including hypersecretion disorders of the meibomian glands (chalazion), sebaceous glands (hordeola) and sweat glands (hyperhydrosis). Chalazion is a chronic granulomatous enlargement of a meibomian gland of the eyelid. This disorder is characterized by hypersecretion of meibum from the meibomian glands. This hypersecretion leads to an accumulation of fatty materials that form lesions that occlude the ductal elements of the gland, leading to an encroachment of the occlusion into the surrounding tissue, which further induces an inflammatory response. Similarly, hordeola is characterized by hypersecretion of sebum from sebaceous glands. Individuals suffering from Chalazia and/or hordeola are often treated by warm compresses or lid soaps which mechanically remove the excess secretion. This approach is often ineffective. The use of antibacterial eyedrops are occasionally effective, but rarely cure the underlying problem—hypersecretion of the meibomian and sebaceous glands that causes inflammation. Patients usually undergo multiple surgical procedures to remove fatty secretions and associated inflammatory cells within the glands to effect relief. Such procedures are painful and occasionally result in lid scarring and misdirection of the eyelashes. The present invention, however, provides an improved method of treating subjects suffering from Chalazion, hordeola and cutaneous infections, comprising the administration of botulinum toxin to reduce or prevent the secretion of meibum and sebum from meibomian and sebaceous glands, respectively.

Chalazia occurs as a chronic deep inflammation of the lid associated with the accumulation of lipid material within macrophages (epithelioid cells) surrounding meibomian glands within the tarsal plate of the eyelid. The inflammation is characterized as a granulomatous-type inflammation associated with lipid and cellular lesions within soft tissues. In the case of chalazia, the lesions are formed by the secretion of the meibomian glands, the glands which contribute to the outer layers of the tear film covering the ocular surface. Histological analysis of these lesions reveal clear regions representing the lipid material, surrounded by polymorphonuclear leukocytes, plasma cells, giant cells, and lymphocytes.

Hordeola presents a similar pathologic process, however, these lesions occur from occluded sebaceous glands at the extreme of the eyelid margin. The resulting occlusion and excess sebum produces an inflammatory reaction similar to that observed in chalazion.

Chalazion formation has been associated with hypersecretion of the lipid-rich meibum from the meibomian gland. Alterations in the lipid composition of meibomian secretions, including free fatty acid and cholesterol content, have also been linked to Chalazion, producing tear film instability, irritation of conjunctival and corneal epithelium, and increased susceptibility to bacterial and fungal infections. Although numerous organism have been identified in the infections frequently associated with Chalazion, the most common isolated bacteria from blepharitic eyelids include species of Staphylococcus, Corynebacterium, and Proprionibacterium. Staphylococcus aureus has been thought to flourish on hypersecretion of meibomian and related eyelid glands. In summary, the pathophysiology of chalazia and hordeola involves: 1) altered meibomian secretion and hypersecretion; 2) inflammation from secretion backup into soft tissue of the lid; and 3) secondary inflammation.

### K. Depressive Disorders

Depressive disorders encompass the diagnoses of major depression, dysthymia, and atypical depression or depression not otherwise specified (“minor depression”). The different subgroups of depressive disorders are categorized and defined by the Diagnostic and Statistical Manual of Mental Disorders, Fourth Edition (DSM-IV). (American Psychiatric Association. Diagnostic and Statistical Manual of Mental Disorders, 4^{th} Ed., Primary Care Version (DSM-IV-PC). American Psychiatric Association Press, Washington, D.C. 1995). According to the DSM-IV, a diagnosis of “major depression” requires that a patient present with at least five of the following nine symptoms during the diagnostic period: 1) depressed mood most of the day (most acute in the morning); 2) markedly diminished interest or pleasure in nearly all activities (anhedonia); 3) significant weight loss or gain; 4) insomnia or hypersomnia; 5) psychomotor agitation or retardation; 6) fatigue or energy loss; 7) feelings of guilt and worthlessness; 8) impaired concentration and indecisiveness; and 9) recurring thoughts of death or suicide. To support a diagnosis of major depression, a depressed mood or loss of interest (anhedonia) must be one of the five observed symptoms. In contrast, a diagnosis of “atypical depression” or “depression not otherwise specified” (also referred to as “minor depression”), the most common form of depression, requires between 2 and 4 depressive symptoms that are present daily or for most of the day for at least a two week period. Dysthymia is a chronic, low intensity mood disorder characterized by anhedonia, low self esteem and low energy that persists for more than two years, consecutively. Seasonal affective disorder is considered to be a form of major depression characterized by seasonal variation.

Depressive disorders do not include normal emotional reactions, a normal grief reaction or reactions secondary to an organic cause such as a physical illness or drug exposure. As used herein, depressive disorders refer to primary disorders of mood and sleep patterns and not secondary or reaction disorders. Such reactionary disorders occur secondarily to other medical disorders such as hyperhydrosis, cervical dystonia, migraine headache, tension headaches, various pain syndromes, jaw spasms, blepharospasm, strabismus, inflammatory local and systemic diseases, post operative pain syndromes, hemifacial spasms, cancer, myocardial infarction, stroke, degenerative neurological diseases, or any other physical ailment causing an emotional reaction.

### L. Anxiety

Anxiety is a group of disorders characterized by a number of both mental and physical symptoms, with no apparent explanation. Apprehension, fear of losing control, fear of going “crazy”, fear of pending death, impending danger, or uneasiness are among the most common mental symptoms. Common physical symptoms include dizziness, lightheadedness, chest pain, abdominal pain, nausea, increased hear rates or diarrhea. Chronic anxiety, also referred to as Generalized Anxiety Disorder, manifests as persistent worries, fears, and negative thoughts lasting a minimum of six months. Chronic anxiety often results in excessive worry over daily activities, headaches and nausea. Sleep disorders or early awakening, depression, tension, muscle aches and fatigue can all accompany chronic anxiety.

Acute anxiety, or Panic Disorder, comes on as a sudden attack or fear accompanied by symptoms that may resemble a heart attack, such as palpitations, chest pain and dizziness. Shortness of breath, stomach upset, chills, cold sweats, hot flashes, or irrational fears of death can combine with these symptoms to create a terrifying experience for the individual experiencing them. Excessive levels of nor epinephrine are seen to increase the rates of breathing and pulse in panic attack sufferers. Post-traumatic Stress Disorder is also classed as an anxiety disorder, and can be triggered by anyone experiencing or witnessing a deeply traumatic event. Some symptoms of Post-Traumatic Stress Disorder can be anger, depression, emotional numbness, flashbacks, nightmares and a tendency to startle easily.

Phobias, or irrational fears, and Obsessive Compulsive Disorder, a tendency towards repetitive or uncontrollable behavior, are also classed with anxiety disorders. These may co-exist together, as many individuals with obsessive compulsive disorder have phobias of germs or lack of cleanliness and may was their hands or bathe excessively.

Anxiety disorders do not include normal emotional reactions, a normal reaction to stress or reactions secondary to an organic cause such as a physical illness or drug exposure.

### M. Sleep Disorders

Circadian rhythm describes the approximately 24-hour cycles that are generated by an organism. Most physiological systems demonstrate circadian variations. The systems with the most prominent variations are the sleep-wake cycle, thermoregulation, and the endocrine system. Circadian rhythm disturbances can be categorized into two main groups: transient disorders (e.g., jet lag, altered sleep schedule due to work, social responsibilities, illness) and chronic disorders. The most common chronic disorders are delayed sleep-phase syndrome (DSPS), advanced sleep-phase syndrome (ASPS), and irregular sleep-wake cycle. Katzenberg et al. have suggested a genetic correlation (i.e., clock polymorphisms) to circadian rhythm patterns. DSPS is characterized by a persistent inability (more than 6 mo) to fall asleep and awaken at socially accepted times. Once asleep, these patients are able to maintain their sleep and have normal total sleep times. (In contrast, patients with insomnia have a lower than normal total sleep time, due to difficulties in initiating or maintaining sleep.) ASPS is characterized by persistent early evening sleep onset (between 6:00 and 9:00 pm) with an early morning wake-up time, generally between 3:00 and 5:00 am. ASPS occurs much less frequently than DSPS and is seen most commonly in the elderly and in individuals who are depressed.

The neural basis of the circadian rhythm, the suprachiasmatic nuclei (SCN), is located in the anterior ventral hypothalamus and has been identified as the substrate that generates circadian activity. Lesions of the SCN produce loss of circadian rhythmicity of the sleep-wake cycle, the activity-rest cycle, skin temperature, and corticosteroid secretion. Other pacemakers exist that are not located in the SCN. For instance, core body temperature rhythm persists in spite of bilateral ablation of SCN. Furthermore, free-running studies have provided evidence for multiple circadian oscillators. Under free-running conditions, circadian rhythm may split into independent components.

The SCN are the site of the master circadian clock in mammals. The SCN clock is mainly entrained by the light-dark cycle. Light information is conveyed from the retina to the SCN through direct, retinohypothalamic fibers. The SCN also receive other projections, like cholinergic fibers from basal forebrain. Cholinergic afferents and transmission have been shown to be involved in regulation of light-induced circadian rhythms. (Erhardt et al. 2004 The Neuroanantomy of the Circadian Rhythm.).

In the United States, DSPS is common. Approximately 7-10% of patients who complain of insomnia are diagnosed with a circadian rhythm disorder, most often DSPS. The prevalence of DSPS is probably higher than that because the total sleep time is typically normal in patients with DSPS and because patients with DSPS adjust their lifestyle to accommodate their sleep schedule and do not seek medical treatment. In adolescence, the prevalence is approximately 7%. In contrast, true ASPS probably is quite rare. An age-related phase advance, however, is common in the elderly, who tend to go to sleep early and get up early.

The diagnosis of circadian rhythm disorders is based primarily on a thorough social, physical and neurological history. Differentiation of transient disorders from chronic disorders and primary disorders from secondary disorders influences the direction of evaluation and treatment plans. As with all medical and psychiatric histories, the nature of the complaint is the first order of business. In cases of sleeplessness, distinguishing individuals with difficulty initiating sleep from those with difficulty maintaining sleep, those with significant daytime impairment, and those complaining of nonrestorative sleep is important.

Disorders associated with various sleep disorders include narcolepsy, cataplexy, restless-leg syndrome, and sleep apnea. Anxiety disorders do not include normal emotional reactions, a normal reaction to stress or reactions secondary to an organic cause such as a physical illness or drug exposure.

### N. CNS Disorders

The present invention is also directed to methods of using botulinum toxin based pharmaceuticals injected transcutaneously or by any of the routes of administration disclosed herein, to induce a central nervous system depressive effect for the treatment of various CNS disorders. The inventor has found that botulinum toxin exerts a CNS depressive effect in rats injected transcutaneously in the scalp. The injections are not intracranial or directly into the brain, but may include or specifically exclude intrathecal and intraspinal injection or administration. It is hypothesized that transcutaneous administration of botulinum toxin penetrates the blood/brain barrier. The present invention provides methods and compositions for using the botulinum toxin based pharmaceuticals disclosed herein for the treatment of seizures, depression, anxiety, agitation, mania, bipolar disorders, generalized seizures, mental retardation, delirium, hyperactivity syndrome, attention deficit disorder (ADD), dementia, Huntington's disease, Alzheimer's disease, Parkinson's disease, psychosis, ALS (Amyotrophic Lateral Sclerosis), also known as Lou Gehrig's Disease, schizophrenia, stroke protection (also known as neuroprotection), glutamate exociticity, head injury, brain hemorrhage, brain aneurysm, metabolic intoxications, insomnia, sleep disorders, and other CNS disorders.

In general, the methods of the invention are directed to the steps of treating any of the disorders mentioned herein by first identifying a subject with the disorder or at least one symptom of the disorder; and administering an effective amount of a composition disclosed herein to said subject to thereby reduce or treat at least one symptom of the disorder or treat the disorder. The methods of the invention

In certain embodiments, the botulinum toxin based pharmaceuticals disclosed herein are used at various dosage levels to induce a generalized atrophic effect in the CNS. This effect is useful in the treatment of various CNS disorders. The inventor has found that rats injected with high doses of botulinum toxin (i.e. doses at or near the LD₅₀) exhibit expanded or enlarged lateral ventricles in their brains. Controls show no such effects while treated animals show a marked effect. Generalized brain atrophy is indicative of biological activity at the level of neurotransmitters that is induced by transcutaneous administration of botulinum toxin. The evidence is consistent with a suppressive effect in the hypothalamus in the treated animals. This could cause direct effects on the release of hormones such as thyroid releasing factors, gonadotropin releasing factor, etc.

All books, articles, patents or other publications and references are hereby incorporated by reference in their entireties. Reference to any compound herein includes the racemate as well as the single enantiomers.

### EXAMPLES

The following Examples serve to further illustrate the present invention and are not to be construed as limiting its scope in any way.

### Example 1

A 78-year-old male who noted sleep disturbances and anxiety was initially diagnosed with blepharospasm. Botulinum toxin was administered by injection, and the subject noted improved sleep and reduced anxiety.

### Example 2

A 44-year-old bus driver was diagnosed with hemifacial spasm and reported symptoms of anxiety. Botulinum toxin was administered by injection. The subject noted a better ability to cope with work-related stresses and cope with difficult situations with less stress.

### Example 3

A 72-year-old consultant diagnosed with hemifacial spasm who reported sleep disturbances and anxiety was treated with botulinum toxin that was administered by injection. The subject reported improved sleep and reduced anxiety and less agitation.

### Example 4

A 45-year-old woman was treated for cosmetic indications with botulinum toxin. The initial diagnosis was cosmetic rhytides. The subject noted fewer symptoms of depression and less anxiety for a period of two months.

### Example 5

A 44-year-old woman diagnosed with severe tension headaches and sleep disturbances was treated with botulinum toxin by injection. The subject noted improved sleep patterns and fewer headaches up to two months after treatment.

### Example 6

A 73-year-old male with essential blepharospasm reported sleep disturbances and anxiety characterized as “nervous tension.” Botulinum toxin was administered by injection. The subject noted less anxiety and improved sleep after the injections. The reduced symptoms lasted two to three months and ultimately recurred.

### Example 7

A 43-year-old person with myofacial pain and sleep problems was treated with botulinum toxin by injection. The subject noted better sleep patterns after injections that lasted three months.

### Example 8

A 42-year-old person was diagnosed with myofacial pain, tension headaches and depression and treated with botulinum toxin administered by injection. The subject noted some improvement in sleep pattern after the toxin injections.

### Example 9

The subject is a 54-year-old person diagnosed with essential blepharospasm and depression. Botulinum toxin was introduced by injection. The subject noted fewer symptoms of depression after the botulinum toxin injections.

### Example 10

The subject is a 57-year-old physician diagnosed with essential blepharospasm. Botulinum toxin was introduced by injection. The subject noted a feeling of euphoria, well being and improved mood after the botulinum toxin injections.

### Example 11

A 47 year old woman with a history of cervicogenic headache and frequent problems of insomnia. The insomnia was characterized by difficulty initiating sleep, intermittent awakening, early-morning awakening, and inability to maintain sleep. Injections were given in the regions generally used to treat spasmodic torticollis as well as in multiple locations along the hairline, both anterior and posterior. Doses ranged between 5-20 units per subcutaneous injection site with a total dose of 100 U. Within 3-5 days, improvement in the insomnia occurred and lasted between 10-14 weeks. Improvement in each component of her sleep disorder occurred. Recurrence of the sleep disorder occurred after the 10-14 week period.

### Example 12

A 52 year old woman received botulinum injections for the effacement of glabellar rhytides (facial wrinkles). Further injections were given in multiple locations along the hairlines, she also suffered from insomnia with difficulty initiating sleep and sustaining sleep. After injection with botulinum toxin, sleep pattern improved and lasted the duration of about 10-12 weeks. Total dose administered in multiple locations was 30 Units.

### Example 13

A 71 year old man with essential blepharospasm was injected with 60 U divided along the peri-ocular region and the forehead. Improvement in sleep pattern characterized by more continuous sleep was noted after each injection. The benefit lasted about 3 months and has been noted over 3 injection cycles. When brought to the patient's attention, he associated the improvement to the botulinum toxin injections. Insomnia recurred when he felt the time for repeat injection with botulinum toxin.

### Example 14

A botulinum toxin composition is prepared from any immunotype (A-G) consisting of monocomponent neurotoxin molecules free of accessory or complex proteins, containing human serum albumin, and a nanoemulsion, with various charges. The nanoemulsion may contain polymers consisting of any of the following: polyethylene glycol, vegetable oil, a vegetable oil derivative or a monounsaturated or polyunsaturated oil. The pH may be altered in the preparation to enhance permeability. Alternatively, botulinum toxin is prepared from immunotypes A-G consisting of a monocomponent neurotoxin, without a nanoemulsion carrier, albumin and an acidic pH between 1-6 units. The effect on the central nervous system from transcutaneous injection was demonstrated using a rodent animal model typically used for research in neurodegenerative disease (20-30 gram mice). Injections were given over the scalp region with botulinum type A toxin at a dose close and approximating the LD₅₀ for this animal. Surviving animals were subjected to autopsy and serial brain cutting and histologically stained using a standard Nissle formula. Substantial atrophy of basal ganglion and periventriclular cells was noted. Such changes are not usually seen with systemic illness without direct brain pathology. The neuropathologic assessment is that direct suppressant effects do occur within the central nervous system at high dose (close to the LD₅₀ for the animal model). More subtle changes are anticipated and seen at lower therapeutic doses based on clinical observations of efficacy for insomnia, dysmenorrhea, depression and anxiety. The experimentation described herein indicates blockage of neurotransmission usually of excitatory neurotransmitters to the extent that pathologic change occurs in brain structures. The major central nervous system neuroransmitters blocked include glutamate, norepinephrine, acetylcholine. GABA effects are augmented. SNAP-25 is noted to be cleaved throughout the targeted areas.

### Example 15

The effect on the central nervous system from transcutaneous injection was demonstrated using a rodent animal model typically used for research in neurodegenerative disease (20-30 gram mice). Four injections of botulinum toxin (totaling 0.8 LD₅₀ units) were given over the scalp region. Surviving animals were subjected to autopsy and serial brain cutting and histologically stained using a standard Nissle formula. Substantial atrophy of basal ganglion and periventriclular cells was noted. Substantial decrease of cholinergic neurons was noted. Substantial decrease in the amount of choline acetyltransferase was noted. More subtle changes are anticipated at lower therapeutic doses based on clinical observations of efficacy for insomnia, dysmenorrhea, depression and anxiety. The experimentation described herein demonstrates blockage of neurotransmission usually of excitatory neurotransmitters to the extent that pathologic change occurs in brain structures. The major central nervous system neuroransmitters blocked include glutamate, norepinephrine, and acetylcholine. GABA effects are augmented. SNAP-25 is noted to be cleaved throughout the targeted areas.

### Example 16

The effect on the central nervous system from transcutaneous injection was demonstrated using a rodent animal model typically used for research in neurodegenerative disease (20-30 gram mice). Four injections of botulinum toxin (totaling 0.8 LD₅₀ units) were given over the scalp region. Surviving animals were subjected to autopsy and serial brain cutting and histologically stained using a standard Nissle formula. Serial cut mouse tissue sections were stained for Nissle substance using cresyl violet and immunostained for glutamate receptor activity. Sections were rinsed in TRIS-buffered saline with Tween 20 (TBS-T) containing 10% normal goat serum for one hour. Sections were then incubated overnight in TBS-T with 0.1% sodium azide and anti-GluR4. Sections were rinsed three times in TBS-T, followed by a 2-3 hour incubation in TBS-T containing a goat anti-mouse peroxidase-conjugated secondary antibody to detect glutamate. Sections were then rinsed three times in TBS-T. Antibody complexes were visualized using diaminobenzidine. Preabsorbtion with excess target protein, or omission of either primary or secondary antibody, were used to demonstrate antibody specificity and background generated from the detection assay. Tissue sections were examined using a Nikon Eclipse E800 microscope with a Spot® digital camera. Photographs of tissue sections of neostriatum in an untreated mouse (sham injection) and a botulinum toxin treated mouse (four injections totaling 0.8 LD₅₀ BOTOX® injected transdermally over the scalp reason).

We claim:

1. An albumin-free composition comprising a botulinum toxin and a hyaluronidase.

2. The composition of claim 1, further comprising a stabilizing sugar.

3. The composition of claim 2, wherein said stabilizing sugar is a saccharide, disaccharide, polysaccharide or oligosaccharide.

4. The composition of claim 3, wherein said stabilizing sugar is trehalose, sucrose or lactose

5. The composition of claim 1, wherein the botulinum toxin is immunotype A, B, C, D, E, F, or G.

6. The composition of claim 5, wherein the botulinum toxin is botulinum toxin type A.

7. The composition of claim 6, wherein the botulinum toxin is from Hall strain Clostridium botulinum.

8-12. (canceled)

13. The composition of claim 1, wherein the hyaluronidase is recombinantly produced.

14-17. (canceled)

18. The composition of claim 1, wherein the hyaluronidase is present in an amount sufficient to stabilize the botulinum toxin.

19-29. (canceled)

30. A method for muscle denervation comprising the step of administering the composition of claim 1 to a subject in need thereof in an amount sufficient to produce local muscle denervation.

31. A method for treating neuromuscular diseases comprising the step of administering the composition of claim 1 to a subject in need thereof in an amount sufficient to produce muscle weakness.

32. A method for treating pain comprising the step of administering the composition of claim 1 to a subject in need thereof in an amount sufficient to reduce pain.

33. A method for cosmetically modifying soft-tissue features comprising the step of administering the composition of claim 1 to a subject in need thereof in an amount sufficient to modify said features.

34. A method for treating inflammation comprising the step of administering the composition of claim 1 to a subject in need thereof in an amount sufficient to reduce inflammation.

35-38. (canceled)

39. A method of treating migraine headache comprising the step of administering the pharmaceutical composition of claim 1 to a subject in need thereof, wherein administration of said formulation reduces migraine headache pain.

40-49. (canceled)

50. A method of treating tension headache comprising the step of administering the pharmaceutical composition of claim 1 to a subject in need thereof, wherein administration of said formulation reduces pain.

51-64. (canceled)

65. A method of treating depression comprising the steps of:
a) identifying a subject with a depressive disorder or identifying a subject with one or more symptoms of a depressive disorder; and
b) administering an effective amount of a composition of claim 1 to said subject thereby reducing at least one symptom of depression.

66-73. (canceled)

74. A method of treating anxiety comprising the steps of:
a) identifying a subject with an anxiety disorder or identifying a subject with at least one symptom of an anxiety disorder; and
b) administering an effective amount of a composition of claim 1 to said subject thereby reducing at least one symptom of anxiety.

75-102. (canceled)

103. A method of delivering a botulinum toxin across a blood-brain barrier, comprising the steps of identifying a subject with at least one symptom of a neuropsychiatric disorder and administering a composition of claim 1 to said subject in an amount sufficient to deliver said neurotoxin across the blood brain barrier.

104-170. (canceled)

171. A method of treating a disorder selected from the group consisting of seizures, depression, anxiety, agitation, mania, bipolar disorders, generalized seizures, mental retardation, delirium, hyperactivity syndrome, attention deficit disorder (ADD), dementia, Huntington's disease, Alzheimer's disease, Parkinson's disease, psychosis, ALS (Amyotrophic Lateral Sclerosis), also known as Lou Gehrig's Disease, schizophrenia, glutamate exociticity, head injury, brain hemorrhage, brain aneurysm, metabolic intoxications, insomnia, sleep disorders, comprising the step of administering to a subject in need thereof an effective amount of the composition of claim 1.
